(19) United States
(12) Patent Application Publication
(10) Pub. No.: US 2005/0014143 A1
(43) Pub. Date: Jan. 20, 2005

(54) Method for determining the existence of animal or vegetable mixtures in organic substrates

(76) Inventors: Carole Donne-Gousse, Bois-Guillaume (FR); Vincent Laudet, Lyon (FR); Catherine Hanni, Lyon (FR)

Correspondence Address: YOUNG & THOMPSON, 745 SOUTH 23RD STREET, 2ND FLOOR, ARLINGTON, VA, 22202, US

(21) Appl. No.: 10/480,587

(22) PCT Filed: Jun. 13, 2002
(86) PCT No.: PCT/FR02/02030

(30) Foreign Application Priority Data
Jun. 13, 2001 (FR) 01/07736

(51) Int. Cl. C12Q 1/68

(52) U.S. Cl. 435/6

(57) ABSTRACT

The invention relates to the use of a cloning method for determining the existence and the identification of a mixture of organic origin containing DNA from different animal species and/or different vegetable species and/or different human individuals.

The invention relates to a method for determining the existence of mixtures of animal or vegetable origin in organic substrates.

More specifically, the invention relates to a method for determining the existence of mixtures of species, populations or races of animal or vegetable origin or of human individuals in organic substrates.

Numerous fields currently use the amplification method (Polymerase Chain Reaction: PCR) in molecular biology as a means of detecting and identifying species. In practice, this technique is commonly used by laboratories engaged in molecular biology, veterinary inspection laboratories, laboratories engaged in the microbiological analysis of foodstuffs, the environment and pharmaceuticals, and laboratories working in the field of genetic fingerprinting. However, if several species or populations or races co-exist in a same sample, there is no known technique on the market by which it can be established, firstly, whether a mixture is present or not and, secondly which enables the species, populations, races or individuals present in this mixture to be specified without having primers specific to all the species, populations, races or individuals present in this mixture. In practice, situations often arise in which analyzed samples are not made up of a single species, population or race. For example, food preparations may be prepared from several species, populations or races (ready-cooked meals, soup, pâté, terrine . . . ). This mixture might be specified by the manufacturer but it may also have been adulterated. In practice, it may well be that this mixture contains more of one species that is cheaper than another species which should have been the only species contained in the preparation (example: salt cod prepared with a small quantity of common cod (Gadus morhua) and a large quantity of Pacific cod (Gadus macrocephalus), which is less expensive.

Faced with the absence of a satisfactory system of detecting and identifying mixtures of species, populations or races of animal and vegetable origin in organic substrates, the applicant has sought to develop a sensitive and reliable method for dealing with this problem.

The objective of the invention is to propose a method for determining the existence of mixtures of DNA from different animal and/or vegetable species, populations or races and/or different human individuals.

Another objective of the invention is to propose a method which enables the DNA of different animal/and or vegetable species, populations or races and/or different human individuals to be detected and/or identified.

The possibility of being able to establish the existence of DNA mixtures from different human individuals is of particular interest to the field of crime detection. In effect, it is currently very difficult to come up with a reliable interpretation of a genetic fingerprint if several individuals are present. The genetic fingerprinting methods currently available, based on the amplification of short repeated and variable sequences (repetitions of CA or micro-satellites) of the nuclear genome, do not enable a definitive conclusion to be reached as to whether a mixture is present. By amplifying variable fragments of the human mitochondrial genome by PCR, the invention now makes it possible to determine categorically whether a sample contains the DNA of several individuals. Quite remarkably, the sensitivity of the methods developed as part of the invention is such that it enables mixtures of human DNA to be detected even if the proportions of each individual are very uneven (90% of one and 10% of the other, for example). In such a situation, genetic fingerprinting methods are often not able to provide any indication that a mixture is present, which means that their results in terms of identifying a specific individual may be false, thus leading to misinterpretation. Sensitive and reliable detection of the existence of a mixture therefore represents a crucial piece of information in interpreting data from genetic fingerprinting.

In its most general form, the invention relates to the use of a cloning method for determining the existence of a mixture of organic origin containing mitochondrial or chloroplast DNA of different animal species, populations or races and/or different vegetable species, populations or races and/or different human individuals.

The method proposed by the invention of detecting a mixture of several species is simple and exhaustive.

The existence of a mixture of different species is determined by a cloning method. This cloning method is commonly used in molecular biology as a means of obtaining a series of identical molecules (nucleic acids) or isolated cells from single-cell organisms (e.g.: bacteria) and multi-cell organisms (e.g.: mammals). Cloning also offers a means of understanding the role and function of numerous genes by manipulating them either to produce them in large numbers or by transferring them from one organism to another.

The expression “DNA of different animal species, populations or races and/or different vegetable species, populations or races” denotes the specific DNA of each of the animal and/or vegetable species, populations or races present in the mixture.

This DNA represents any nucleotide sequence from the nuclear, mitochondrial or chloroplast genome, and provides a means of making a reliable distinction between the different species or populations or races studied or the different individuals studied. A species is defined as being a group of living organisms, genetically separate from other living organisms and capable of reproduction exclusively within that group.

A population is a group of individuals of one species which can be distinguished from other individuals of the species on the basis of specific morphological and/or genetic characteristics. A species therefore often has several distinct populations. A race is a group of individuals from one domestic species, obtained following lengthy selection to improve a specific trait (such as milk-producing ability in the case of cows, or the quality of meat for pork) and which can be distinguished from the other individuals of this domestic species by specific morphological or genetic characteristics.

The expression “DNA of different human individuals” refers to any mitochondrial or nuclear DNA sequence of human origin which enables a distinction to be made between two individuals. It is the general view that a sequence must contain a minimum of 1 difference for every 100 nucleotides if it is to be regarded as reliable.

By “mixture of organic origin” is meant a mixture containing the DNA of different species, populations or races from tissue taken from living creatures, either animal or vegetable. Organic matter is understood as meaning any solid or liquid matter which can be assumed to be at least partially organic in origin.

By virtue of one advantageous embodiment, the invention relates to the application described above and is characterized by the fact that the mitochondrial or chloroplast DNA is degraded.

Degraded DNA is characterized by its appearance following extraction based on the method described above and deposit on a 1% agarose gel stained with ethidium bromide. It has been observed that DNA taken from fresh tissue takes the form of large molecules (i.e. approximately 20 kbp; see FIG. 5 channel 1) whereas degraded DNA extracted from tissue that has been subjected to extensive alteration such as cooking is either barely visible in the form of a spread of small molecules (100 to 500 bp; see FIG. 5 channel 3) or totally invisible (see FIG. 5, channels 4 and 5). Accordingly, only positive amplification by PCR is able to reveal the presence of this DNA.

In another advantageous embodiment, the invention relates to the use of a cloning method for detecting and/or identifying the DNA of each of the different animal species, populations or races and/or each of the different vegetable species, populations or races and/or each of the different human individuals present in the mixture of organic origin containing DNA of animal species and/or vegetable species and/or human individuals.

By “detection of the DNA of each of the species, populations or races or of each of the human individual” is meant the fact of discerning the presence of DNA specific to each of the species or populations or races or individuals, so as to be able to evaluate the complexity of the initial mixture and, during a second stage, identify the different components of this mixture.

By “identification of the DNA of each of the different species, populations, races or of each of the individuals” is meant the fact of analysing the DNA of each of the species or populations or races or individuals, having detected the presence of species on the basis of their specific DNA, so as to deduce therefrom the composition of the initial mixture.

In one advantageous embodiment of the invention, the cloning method is used in such a way that each animal species, population or race and/or each vegetable species, population or race and/or each human individual is represented by at least one DNA sequence or at least one DNA fragment, and in particular by a unique DNA sequence or a unique DNA fragment, each of said DNA sequences or each of said DNA fragments being taken from the DNA extracted from the mixture of organic origin.

In the description above and in that which follows, the expression “at least one DNA sequence or at least one DNA fragment” means either that there are several copies of the same fragment or the same sequence of DNA or that there are several copies of different fragments or different sequences of DNA.

In one advantageous embodiment of the invention, the cloning method is used as a means of detecting and/or identifying the DNA of each of the different animal species present in a mixture of animal species, and in particular the DNA of each of the sub-species, lineages, races, varieties, strains and/or populations present in said mixture, each of said animal species, and in particular each of said sub-species, races, varieties and/or strains, being represented by at least one DNA sequence or at least one DNA fragment, and in particular by a unique DNA sequence or a unique DNA fragment.

By “animal species, sub-species, lineages, races, varieties, strains and populations” is meant groups or sets of animals from a same species which resemble one another on the basis of certain criteria. Sub-species and populations may be regarded as synonymous with one another. A sub-species is a population which can be specifically identified and is known by a specific Latin name. For example, Otus megalotis everetti and Otus megalotis nigrorum are two different sub-species of the species Otus megalotis, a bird of prey. The terms races, varieties and strains are primarily used to denote domestic animals and may be regarded as synonymous.

In another advantageous embodiment of the invention, the cloning method is used as a means of detecting and/or identifying the DNA of each of the different vegetable species present in a mixture of vegetable species, and in particular the DNA of each of the sub-species, lineages, races, varieties, strains, cepage and/or populations present in said mixture, each of said vegetable species, and in particular each of said sub-species, races, varieties and/or strains being represented by at least one DNA sequence or at least one DNA fragment, and in particular by a unique DNA sequence or a unique DNA fragment.

By “vegetable species, sub-species, lineages, races, variety, strains, cepage, populations” is meant groups or sets of vegetables from a same species which resemble one another on the basis of certain criteria. “Cepage” is the term specifically used to denote a strain or a race or a variety of vine of the Vitis type.

In another advantageous embodiment of the invention, the cloning method is used as a means of detecting and/or identifying the DNA of each of the different human individuals present in a mixture of DNA from different human individuals, each of said human individuals being represented by at least one DNA sequence or at least one DNA fragment, and in particular by a unique DNA sequence or a unique DNA fragment.

By “human individuals” is meant any of the members of the species Homo sapiens.

In one advantageous embodiment of the invention, the DNA extracted from the mixture of organic origin is old (fossil) DNA, degraded DNA or modern DNA.

By “old DNA” is meant a DNA extracted from an organism after its death, which has already undergone a process of degradation. Old DNA very often exhibits signs of degradation, such as, for example, the presence of small DNA molecules, of a size less than or equal to approximately 200 base pairs.

By “degraded DNA” is meant DNA which has undergone deterioration caused by transformation processes. Degraded DNA generally takes the form of small fragments (less than or equal to approximately 200 bp) and in a small quantity.

By “modern DNA” is meant DNA extracted from a living individual or extracted very shortly after his death. Modern DNA is characterized by the presence of very large fragments of more than 20 000 base pairs.

In another advantageous embodiment of the invention, the DNA extracted from the mixture of organic origin is:
non-degraded DNA, in particular from a fresh organic sample or
degraded DNA, in particular from an organic sample which has been processed, in particular cooked, freeze-dried, dried, preserved in brine, canned or pasteurized.

By “non-degraded DNA” is meant a DNA which has not undergone any deterioration and is therefore intact. Provided the extraction conditions are right, such DNA is present in the form of molecules with at least 20 000 base pairs.

Examples of situations in which DNA is found in degraded form are:
foodstuffs (cooked, freeze-dried, dried, preserved in brine, canned, pasteurized . . . )
fertilisers, flours, seeds (crushed, dried, fermented . . . )
egg shells, bones, teeth, fur, hair, feathers, excrement (drying, effect of time or of temperature . . . )
alcohols (rendered alcoholic, distilled, fermented . . . )
leathers, skins, pelts, mummified tissue (tanned, preserved and stuffed, dyed . . . )
parchments, papers, wood (effect of time, transformation process used in paper-making . . . )
ivory, amber (effect of time . . . )
glues, natural pigments (paints), grounds, sediments (processing methods used in these industries)
corpses and human or animal bones (effect of time or the environment).

In another advantageous embodiment of the invention, the cloning method is used as a means of detecting and possibly identifying the DNA of each of the different animal species, populations or races and/or each of the different vegetable species, populations or races and/or each of the different human individuals present in the mixture of organic origin, in which at least one of said species, populations or races and/or at least one of said individuals is represented by an old DNA sequence or a degraded DNA sequence or an old DNA fragment or a degraded DNA fragment.

In another advantageous embodiment of the method proposed by the invention, the cloning method is preceded by a stage during which each of the DNA sequences or each of the DNA fragments characteristic of each of the animal species, populations or races and/or each of the vegetable species, populations or races and/or each of the human individuals whose DNA sequences or DNA fragments are to be detected is amplified, the amplified DNA sequences or amplified DNA fragments obtained from the amplification process being contained in a unique amplification product.

Analysing the genome by means of nucleic probes is an efficient way of identifying species but is not very far advanced at present. Work conducted on old DNA (or fossil DNA) since the early 1990s has shown that DNA is a very stable molecule after death, in spite of the effects of time and the environment (Brown and Brown., 1994, Bioessays 16:719-26). However, where it continues to exist in old substrates, this DNA is very much degraded and present in only small quantities in molecular forms that are damaged and chemically modified (P\ddot{aa}bo, 1989, Proc. Natl. Sci USA 86: 1939-1943). These characteristics are essentially due to the phenomena of hydrolysis and oxidation (Lindahl, 1993, Nature 362:709-715). Thanks to the PCR technique (Polymerase Chain Reaction), a remarkable and powerful analytical tool, it is possible to multiply a given fragment of DNA “in vitro” on an almost exponential basis. By amplifying the DNA of a food or any other preparation which has undergone modification such as that due to cooking, smoking etc., it will be possible to identify components of animal or vegetable origin. For example, PCR has recently been used as a means of characterising cooked pork (Meyer et al., 1994, Journal of the AOAC International 77(3), 617-622), sheep or goat meat (Chikuni et al., 1994, Meat Science 37(3) 337-345). Likewise, amplifying specific sequences of the Y chromosome has made it possible to determine the sex of butchered carcasses of bovine and ovine origin (Apparao et al., 1995, Meat Science 39(1), 123-126). The resultant sequences are analyzed by molecular phylo-engineering, which enables different species to be identified (Wolf, 1999, J Agric Food Chem 47: 1350-1355).

In practice, the invention is based on determining a mixture. In technical terms, this can be done at different levels. It is done firstly by “direct sequencing” of the amplification product, containing the fragment or fragments of DNA, and observing the electrophoretic micrographs obtained. Illegibility of the sequence may be interpreted as being:
either due to degradation of the DNA if it is in too poor a state to be analyzed or
alternatively a mixture of at least two species if a superposition of sequences is observed on a level with “indeterminate peaks” represented by the letter N.

Secondly, the characteristics of a mixture can be determined once the amplification product has been cloned. If this amplification product contains only a single fragment of DNA, there is only one species, in which case a sample of 10 bacterial colonies will produce 10 identical sequences. If, on the other hand, a mixture of species is present, there will be as many different sequences as there are species present in the sample.

Advantageously, the DNA sequence is of mitochondrial origin in the case of animals and vegetables or is chloroplast DNA in the case of vegetables. Mitochondrial DNA (mtDNA) has proved to be the most suitable molecule for this type of analysis (Kocher et al., 1989, Proc. Natl. Acad. Sci. USA 86:6196-6200). From a technical point of view, it is easier to detect than genomic DNA because it is present in 100 to 1000 copies per cell compared with two copies in the case of nuclear DNA. It can therefore be more reliably detected in organic matter in which the DNA has been exposed to various physical (temperature, pressure etc.) and chemical or biochemical factors leading to its degradation. Furthermore, it is an excellent species marker and is often used in phylo-engineering. In effect, depending on the species studied, certain regions of mtDNA enable species to be distinguished from one another, whilst others have a capacity for even finer resolution and enable different populations to be distinguished (geographic races, sub-species and even individuals): control region for mtDNA replication, cytochrome b, cytochrome c oxidase or mitochondrial RNA (RNA 12S or RNA 16S).

In one advantageous embodiment of the invention, each amplified DNA sequence or each amplified DNA fragment is of nuclear or mitochondrial origin if the aim is to detect and/or identify a mixture containing the DNA of different animal species and/or different human individuals, whereas it is of nuclear, mitochondrial or chloroplast origin if the aim is to detect and/or identify a mixture containing DNA from different vegetable species.

The invention relates to a method for detecting and/or identifying the DNA of each of the different animal species, populations or races and/or each of the different vegetable species, populations or races and/or each of the different human individuals present in a mixture of organic origin, each of said animal species, populations or races and/or each of said vegetable species, populations or races and/or each of said human individuals being represented by at least one DNA sequence or at least one DNA fragment, in particular a unique DNA sequence or a unique DNA fragment, each of said DNA sequences or each of said DNA fragments being DNA taken from the DNA extracted from said mixture, said method being characterized in that it comprises the following stages:
amplification of each of the DNA sequences or each of the DNA fragments characteristic of the animal species, population or race, of the vegetable species, population or race or of the human individual to be detected and/or identified, in particular by the polymerase chain amplification method (PCR), in order to obtain a unique amplification product containing the different amplified DNA fragment(s) or the different amplified DNA sequence(s),
cloning the amplification product obtained from the preceding amplification stage in order to separate the different DNA sequences or the different DNA fragments present in the mixture of organic origin,
sequencing each of the DNA sequences or each of the DNA fragments thus separated from said mixture.

The invention further relates to a detection and/or identification method as defined above, in which:
the polymerase chain amplification method (PCR) consists in repeating the cycle of the following stages:
heating of the DNA extracted from the mixture of organic origin in order to separate the DNA into two single-chain strands,
hybridization of the single-chain DNA strands at an appropriate temperature with the appropriate oligonucleotide primers in order to amplify the DNA of animal or vegetable species, populations or races, or of the human individuals to be detected and/or identified,
elongation of said appropriate oligonucleotide primers with a polymerase at an appropriate temperature to obtain a unique amplification product containing each of said DNA sequences or each of said DNA fragments characteristic of a given animal or vegetable species, population or race, or of a human individual,
cloning the amplification product obtained from the preceding amplification stage, comprising the following stages:
with the aid of a DNA ligase enzyme, inserting said unique amplification product containing the different amplified DNA sequences or the different amplified DNA fragments characteristic of the DNA of the animal or vegetable species, population or race, or of the human individual to be detected and/or identified, in a previously linearized plasmid, in order to obtain several plasmids, each containing a unique amplified DNA sequence or a unique amplified DNA fragment,
incorporating each plasmid obtained from the preceding stage, respectively in a bacterium, in particular Escherichia coli (E. coli),
multiplying each of the bacteria obtained in the preceding stage by cultivating said bacteria in an appropriate medium, in the presence of an antibiotic, in particular ampicillin, in order to select bacteria which have incorporated a plasmid in which a DNA fragment or a DNA sequence has been inserted,
separating each of the plasmids containing a unique DNA sequence or a unique DNA fragment from each of the bacteria containing said plasmids, in order to recover all the plasmids or “plasmid DNA” thus multiplied, each of said plasmids containing a unique DNA sequence or a unique DNA fragment,
taking a sufficient number of plasmid (or “plasmid DNA”) samples from all the plasmid (or “plasmid DNA”) samples obtained from the preceding stage in order to detect and/or identify at least two different DNA fragments or DNA sequences, each of said DNA sequences or DNA fragments being characteristic of a given animal or vegetable species, population or race, or of a human individual.

“Amplification product” as used in the above description refers to the amplified DNA fragment or fragments or amplified DNA sequence or sequences obtained from the polymerase chain reaction (PCR). The amplification product contains several copies of different amplified fragments or different amplified sequences of DNA if the sample of organic matter to be analyzed contains a mixture of different DNA fragments from different animal or vegetable species, populations or races or from different human individuals.

In one advantageous embodiment of the invention, during the detection and/or identification process, each of the plasmid DNA samples obtained from the cloning method is identified by sequencing, which enables the DNA characteristic of each of the animal species, populations or races and/or each of the vegetable species, populations or races and/or each of the human individuals present in the mixture of organic origin to be identified.

The cloning process has never been described as a technique for separating species, populations, races or individuals from a mixture before. Molecular cloning is a basic technique used in molecular biology as a means of selecting a colony of cells containing specific DNA sequences. In the present case, it is used as a means of separating DNA fragments. These fragments are obtained by a global strategy of the type defined below. The choice of primers depends on the task at hand. Generally speaking, the aim is to look for related species which are mixed with one another but are of different costs in commercial terms. For example, the largest possible primers are used, i.e. which can amplify an order, a family or a group of a quite specific species. The DNA extraction process is identical to that described in detail in example 6 below. It is to the amplification product that the cloning process is applied. Accordingly, the starting point will be the resultant amplified fragment obtained in conjunction with the primer pair selected for the global strategy, which is then cloned. In the majority of cases, however, the aim is to detect the mixture and sequencing does not therefore take place before the cloning phase.

An Invitrogen “TOPO TA Cloning” kit can be used for the cloning method, which works on the following principle. The DNA fragments obtained after amplification are purified and then introduced into a plasmid, which has been linearized beforehand by the supplier. Plasmid is a circular DNA molecule, replication of which within a cell takes place independently of the replication of the genome of this cell. Once the DNA fragment has been introduced into the plasmid using the DNA ligase enzyme, the plasmids are introduced into bacteria such as Escherichia Coli (by what is referred to as a “transformation” process, involving an osmotic shock and a temperature shock or an electric shock). The latter are then reproduced and selected by the cloning technique. Plasmids often code for an enzyme capable of chemically degrading or altering an antibiotic substance, conferring on the bacteria into which they have been introduced a degree of resistance to an antibiotic (such as ampicillin). This resistance is used in genetic engineering as a means of selecting the bacteria containing the plasmid, in the presence of antibiotic. It should be pointed out that this resistance to antibiotics is a natural phenomenon. The cells are then cultivated on a box in nutrient medium to produce bacteria, so that each cell replicates to produce a clonal colony. Usually, cultivation takes place in the presence of an antibiotic whose plasmids possess a resistance gene, thereby enabling clones which have not received plasmids to be eliminated. Each colony contains a single type of plasmid. A visual system is used to ascertain which bacteria have incorporated the plasmids, which in turn have incorporated a DNA fragment. Blue colonies and other white colonies grow on the box of gelose. In effect, two reagents (IPTG and X-Gal) are introduced into the gelose-containing medium which will produce a blue colouration in contact with β-galactosidase. If β-galactosidase is synthesized from this enzyme (blue colouration), the DNA fragment is not ligated to the plasmid because if there were ligation, the DNA fragment would have to position itself at the centre of the gene coding for β-galactosidase, thereby blocking its synthesis, which is what causes the white colouration of the bacterial colonies. These colonies are analyzed and the one which contains the sought plasmid is cultivated and amplified. The plasmid is extracted from the bacterium and then sequenced.

The obtained sequences are compared with one another; if two sequences are different, then there are two species present. It should be noted that in most cases, ten clone samples (white colonies) are enough to detect two species. Since the samples are taken at random, it may be that only a single species will occur in the ten clones, whereas the mixture contains at least two. If there is a suspicion that there are more than two species in the mixture, more clone samples can be taken (15, 20 or even 30). Generally speaking, five clones taken at random should be enough to detect one species. As a rule, about ten plasmid DNA samples are therefore recovered from ten independent white bacterial colonies selected at random. These ten plasmid DNA samples are therefore sequenced to obtain ten sequences. These sequences are analyzed in order to ascertain whether there are different patterns. If there is only one species in the DNA extracted from the sample of organic materials, there will therefore be a single sequence pattern for the ten sequenced clones and the species present can be identified by comparing this sequence with the reference sequences. If, on the other hand, several species are present in the DNA extracted from the sample of organic materials, the number of patterns obtained will correspond to the number of species present in the sample. These sequences can be compared with the reference sequences in order to identify what species are present. The number of clones selected may be increased if it is suspected that the number of species to be identified is higher.

This technique may be used for all organic substrates.

For example, in one advantageous embodiment of the method proposed by the invention, the sequencing of each of the different amplified DNA fragments is preceded by a cloning method if the sample of organic matter contains a mixture of different DNA fragments from different animal or vegetable species or from different human individuals, said cloning method enabling said different DNA fragments from different animal or vegetable species or from different human individuals to be separated from the mixture.

The invention further relates to oligonucleotides, which are characterized in that they are selected from those:

1) presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No21 (position 16123 to 16144 of the mitochondrial DNA of salmon—replication control region according to Hurst, et al., 1999. Gene 239: 237-242):

GCC GAA TGT AAA GCA TCT GG

2) or those presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No22 (position 16341 to 16361 of the mitochondrial DNA of the salmon—replication control region—according to Hurst, et al., 1999. Gene 239: 237-242):

ACC TTA TGC ACT TGA TAT CC

3) or those presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No24 (position 14985 to 14996 of the mitochondrial of the salmon—cytochrome b—according to Hurst, et al., 1999. Gene 239: 237-242):

ACC GGG TCT AAT AAC CCA GC

4) or those presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No25 (position 15409 to 15430 of the mitochondrial DNA of the salmon—cytochrome b—according to Hurst, et al., 1999. Gene 239: 237-242):

ATG ATA ATG AAT GGG TGT TC

5) or those presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No28 (position 356 to 375 of the mitochondrial DNA of the salmon—replication control region—according to Albert et al., 1992, Science 257 (5076), 1491-1495):

AAA GCT CTG CGC GCT CTA CG

6) or those presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No29 (position 539 to 558 of the mitochondrial DNA of the salmon—replication control region—according to Albert et al., 1992, Science 257 (5076), 1491-1495):

CCG CGG AGA CAT TCA TAA AC

The invention further relates to primer pairs, which are characterized in that they are constituted by:
the oligonucleotides SEQ ID No21 and SEQ ID No22
the oligonucleotides SEQ ID No24 and SEQ ID No25
the oligonucleotides SEQ ID No28 and SEQ ID No29

The invention further relates to a DNA fragment as amplified using the primer pairs defined above and containing approximately 100 to approximately 500 base pairs.

The invention also relates to a DNA fragment as defined above, characterized in that it presents a sequence identity of at least 80%, preferably 90% and advantageously 95% with at least one of the sequences contained in:

SEQ ID No23 below:

GCCBWWHDWR VVRCAYSTKB BBMWTRVWKH MRATCWYRWT GCSCGTTRMT CRCVRMRYCK
DBCRBYYTHT TRWVYRCHYM BGGKWSYYYT YHWTKYWTYY TTWYCKYHYR GSKKGYMYDY
MCMRRTRSMA BYDMRRRRSK CYVRMRMBSK MRVMCYVKAY STYGMATTCC AGAGARYMYM
TGYVTYWKVK YSMWRYSHYA THCTMTHADK DATYRCWYMH TKRGAYRKYH RARYATAHRG
KBRAT

in which B is C, G or T, W is A or T, H is A, C or T, D is A, G or T, R is A or G, V is A, C or G, Y is C or T, S is C or G, K is G or T, M is A or C,

SEQ ID No26 below:

WCVGGVTCHA AYAACCCMVY AGGHATYWMM TCMSAYKYHG AYAAAATYHC MTTYCACCCH
TACTWYWCMW WYAARGACVY YYTNGGMTTN VYHSYYWTMC THMYYKBHMT RAYAYYMYTA
RYHCTRTTCK CMCCMRACCT CCTMGGVGAC CCRGAHAAYT WYACVCYWGC MAAYCCMYTM
RWHACHCCHC CHCAWATCAA RCCHGARTGA TAYTTYYTAT TYGCMTAYRC MATYYTHCGM
TCMRTYCYAA YAAACTWGGM GGHGTMCTHG CCCTMKYMBY MTCNRTCCTV RTYCTHDYHV
TMRTYCCYHT MCTMCAYAHM TCYAARCAAC RMRSMMTRAY MTTYCGMCCM CTMWSCCAAW
BMYTWTWYTG RVYYCTRGYM GCVRACMTHC TNAYHCTHAC MTGAATYGGR RGVMWACCHG
TVRRMYACCC HTWYAYYAYC ATYGG

in which W is A or T, V is A, C or G, H is A, C or T, Y is C or T, M is A or C, S is C or G, K is G or T, R is A or G, N is A, C, G or T, D is A, G or T, B is C, G or T,

SEQ ID No30 below:

AAAGCTCTGC GCGCTCTACG TCTARAGGAT CTGCGAATCC CCCTGCTTAT ACTAAAACTT
TCCAAGGCCC GCCTCATGGC ATCCAAGTTG AGAGAGATAA ATTGAACAAG TATGGTCGTC
CCCTATTGGG ATGTACTATT AAACCTAAAT TGGGGTTATC CGCTAAGAAC TATGGTAGAG
CWGTTTATGA ATGTCTCCGC GG

in which R is A or G, W is A or T.

### DESCRIPTION OF THE DRAWINGS

FIG. 1: FIG. 1 illustrates the cloning process used in the context of the invention in order to separate the different DNA fragments of different species of fish from a same mixture.

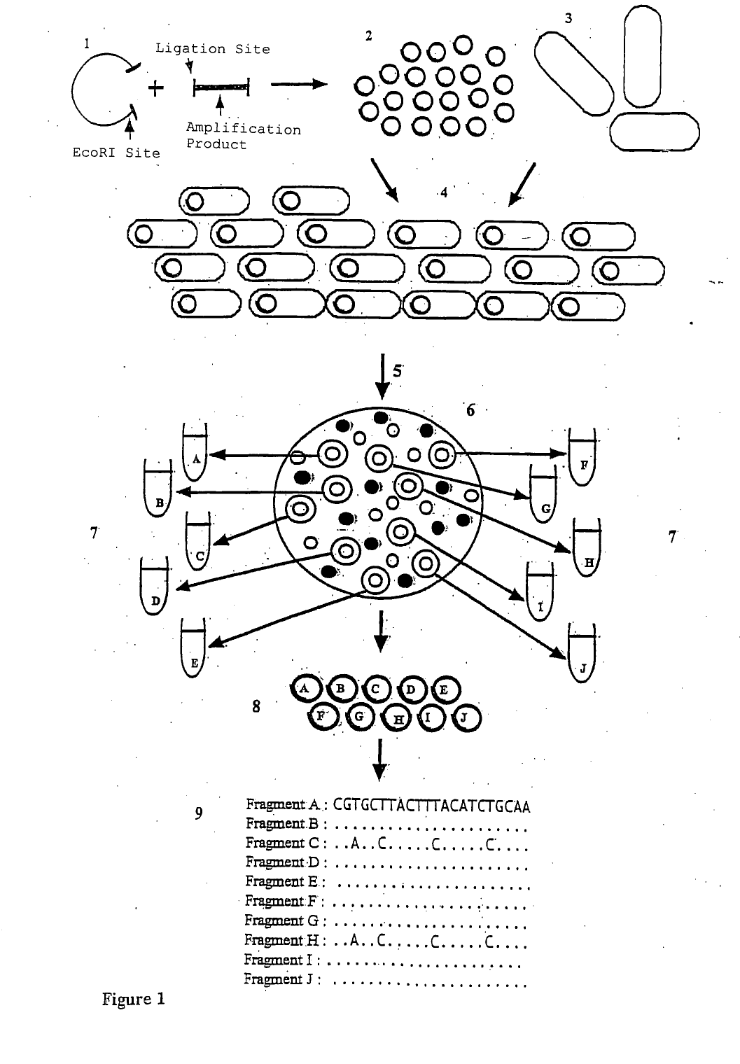

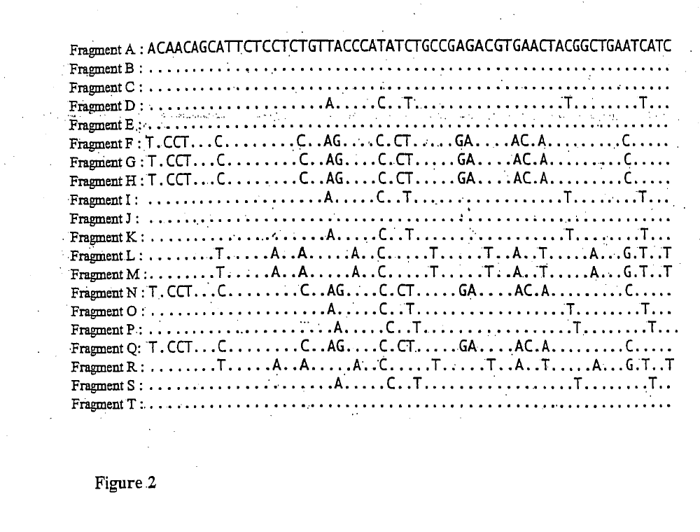

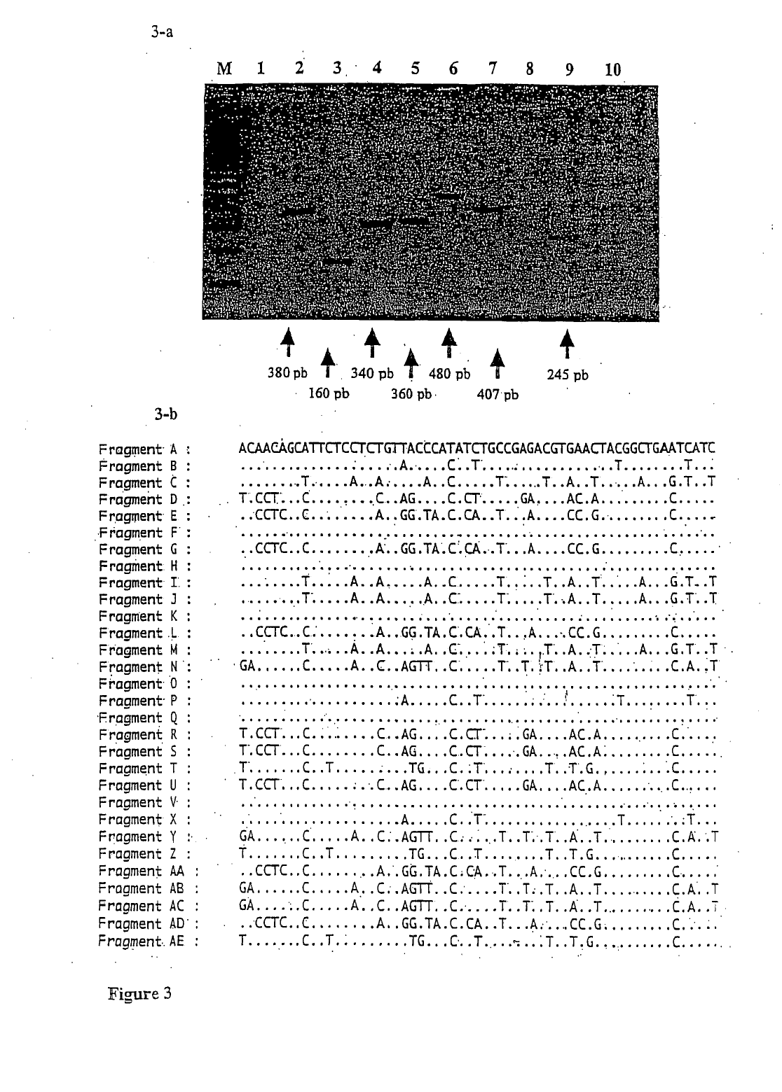

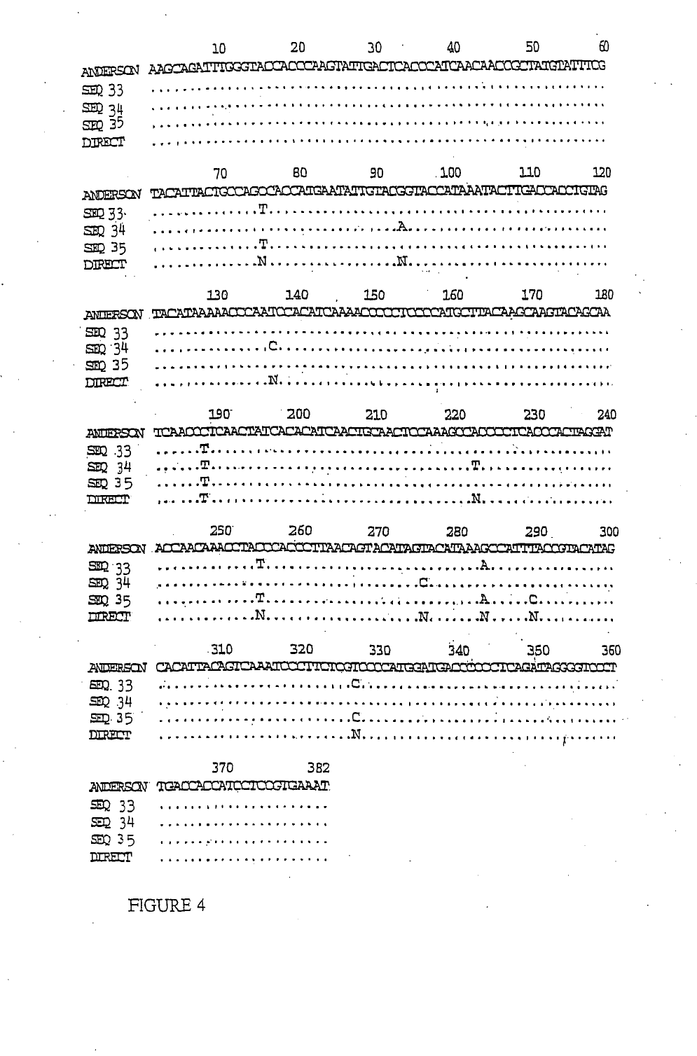

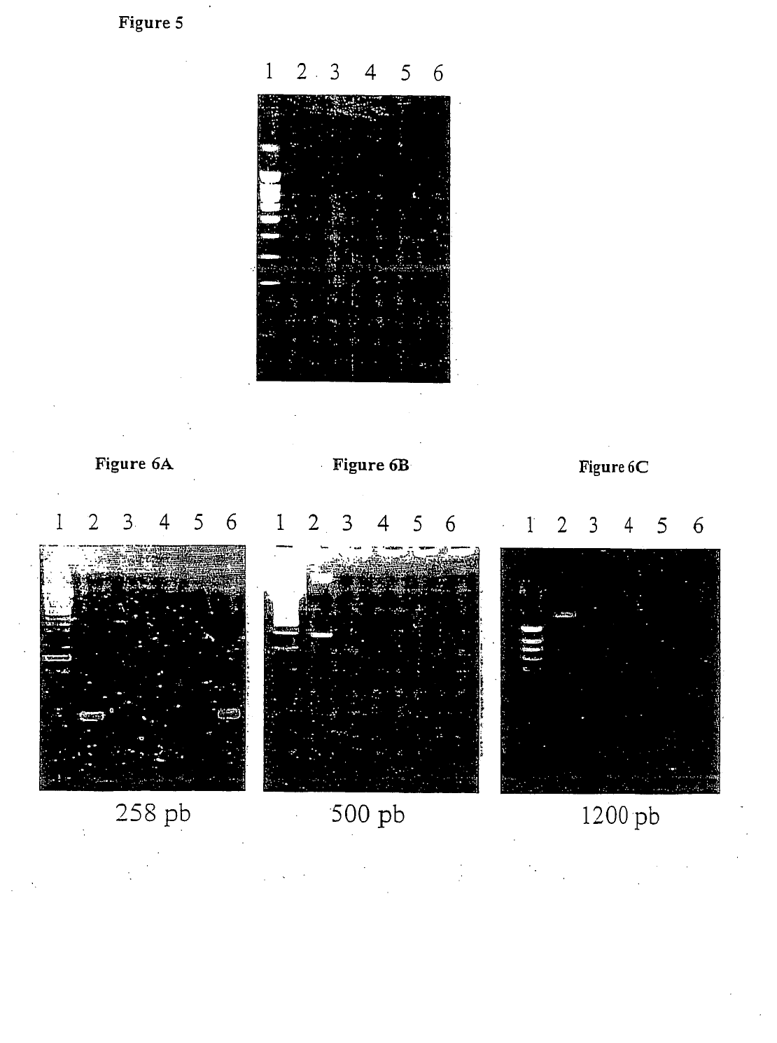

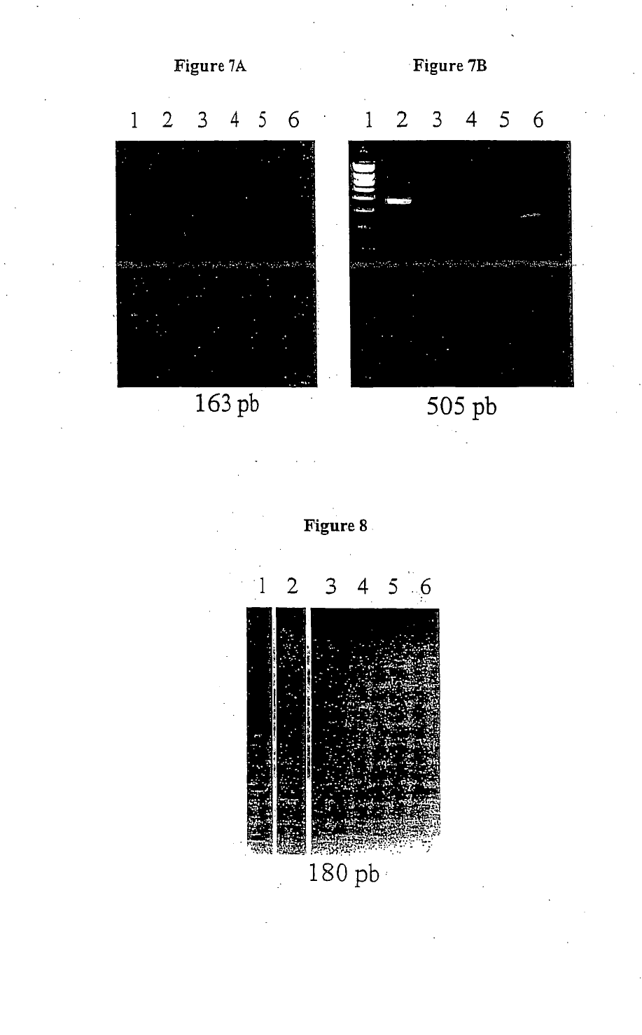

Part 1 of FIG. 1 illustrates the plasmid pCR®2.1-TOPO. The amplification product of the invention, which contains several copies of different DNA fragments is placed in contact with the cut plasmid pCR®2.1-TOPO, with the aid of a DNA ligase enzyme.

Part 2 shows a part of each of the DNA fragments (initially contained in the amplification product) ligated in a plasmid pCR®2.1-TOPO. One plasmid corresponds to each DNA fragment.

Part 3 represents the bacterial cells Escherichia coli (E. coli).

In part 4, the E. coli bacteria are transformed by introducing the plasmids illustrated in part 2. One plasmid corresponds to each E. coli bacterium.

Part 5 (symbolized by an arrow) represents the spread of the E. coli bacteria on a medium containing an antibiotic (for example ampicillin), in order to select bacteria which have incorporated the plasmid.

Part 6 illustrates a gelose box on which blue (symbolize by ●) and white (symbolized by ∘) colonies are grown. The blue colonies are characteristic of the bacterial cells in which the DNA fragments are not ligated with the plasmid, whilst the white colonies are characteristic of the bacterial cells in which the DNA fragments are ligated with the plasmid.

Part 7 illustrates individual samples of colonies of white bacteria (for example 10 bacterial colonies selected at random and denoted by letters A to J) which are individually cultivated because one plasmid corresponds to each colony and hence a specific DNA fragment (denoted respectively by the letters A, B, C, D, E, F, G, H, I and J).

In part 8, the bacteria have been eliminated in order to recover the plasmids and their DNA fragments (A to J). Enough plasmid DNA will therefore be obtained for sequencing purposes.

Part 9 shows the result of sequencing the different DNA fragments, A to J, with the aid of two specific primers of the plasmid bordering the DNA fragment to be sequenced. From the ten sequenced fragments, two different types of sequences are obtained. For example, the analyzed mixture contains two different species of gadiformes, in this particular case Gadus morhua (fragments A, B, D, E, F, G, I and J), and Merluccius hubbsi (fragments C and H).

FIG. 2: FIG. 2 shows the result of sequencing the different DNA fragments A to T, with the aid of two specific primers of the plasmid bordering the DNA fragment to be sequenced. From the 20 fragments sequenced, four different types of sequence are obtained. For example, the analyzed mixture contains four different species of vertebrates, in this particular case Bos taurus (beef: fragments A, B, C, E, J and T), Ovis aries (sheep meat: fragments D, I, K, 0, P and S), Sus scrofa (pork: fragments L, M and R) and Gallus gallus (chicken: fragments F, G, H, N, N and Q).

FIG. 3: FIGS. 3-a and 3-b show the results obtained from the process of detecting and identifying the mixture of species in a sample taken from a can of pet food using two strategies for analysing the species.

In FIG. 3-a, the extracted DNA was amplified with the aid of different primer pairs specific to animal species and deposited on an agarose gel stained with ethidium bromide. A size marker for 100 base pairs (M) is placed on the gel. Well 2 is the amplification product obtained using transvertebrate primers which migrate forming a band of 380 base pairs. Well 3 is the amplification product obtained using primers specific to chicken which migrates forming a band of 160 base pairs. Well 4 is the amplification product obtained using primers specific to gadiformes which migrate forming a band of 340 base pairs. Well 5 is the amplification product obtained using primers specific to Barbary duck which migrates forming a band of 360 base pairs. Well 6 is the amplification product obtained using primers specific to beef which migrate forming a band of 480 base pairs. Well 7 is the amplification product obtained using primers specific to pork which migrates forming a band of 407 base pairs. The primers specific to sheep did not result in an amplification product, which means that there is no sheep in the sample (well 8). Well 9 is the amplification product obtained using primers specific to salmon, which migrates forming a band of 245 base pairs. The primers specific to the rat and mouse did not produce an amplification product, which means that the sample contains no rat or mouse (well 10). Well 1 is a negative amplification reference sample.

In FIG. 3-b, the different DNA fragments A to AE were sequenced using two primers specific to the plasmid bordering the DNA fragment to be sequenced. From the 30 fragments sequenced, seven different types of sequence are obtained. For example, the analyzed mixture contains a mixture of seven species, in this particular case Bos taurus (beef: fragments A, F, H, K, O, Q and V), Sus scrofa (pork: fragments C, I, J and M), Gallus gallus (chicken: fragments B, D, P, R, S, U and X), Cairina moschata (Barbary duck: fragments E, G, L, AA and AD), Salmo trutta (salmon: fragments T, Z and AE) and Theragra chalcogramma (pollack: N, Y, AB and AC).

FIG. 4: shows the alignment of a part of the region which controls human mitochondrial DNA, permitting individual identification (Anderson et al. “Sequence and organization of the human mitochondrial genome” Nature 1981 vol 290, 457-465). SEQ ID No 33, SEQ ID No34 and SEQ ID No35 are the different clone sequences obtained after amplification in respect of the oligonucleotides corresponding to SEQ ID No31 and SEQ ID No32. DIRECT corresponds to the direct sequence of the PCR product.

FIG. 5 shows a DNA extraction carried out from fresh beef DNA (channel 2), or different substrates containing degraded DNA taken from “corned beef” (channel 3), ravioli (channel 4) or potato hash (channel 5). Channel 6 contains the blank extraction (DNA extracted without substrate) and channel 1 contains the size marker.

FIGS. 6A, 6B and 6C show the amplification of fragments of mitochondrial DNA (control region and cytochrome b) taken from mitochondrial DNA extracted from samples of fresh beef (non-degraded mitochondrial DNA; channels 2), or substrates containing degraded mitochondrial DNA taken from ravioli (channels 6). Channels 1 contain the size marker and channels 3, 4 and 5 represent the PCR carried out from blank extraction in order to check for any contamination of the extraction reagents, amplification reagents or the environment.

In FIG. 6A, the oligonucleotides used, namely the sequences represented by SEQ ID NO: 3 and SEQ ID NO: 12, enable a fragment of 258 bp to be amplified.

In FIG. 6B, the oligonucleotides used, namely the sequences represented by SEQ ID NO: 3 and SEQ ID NO: 6, enable a fragment of 500 bp to be amplified.

In FIG. 6C, the following oligonucleotides were used:

| | |
|---|---|
| H1: | 5′ TCA TCT CCG GTT TAC AAG AC 3′ |
| and | |
| L2: | 5′ TGA TAT GAA AAA CCA TCG TTG 3′ |

These oligonucleotides enable a fragment of 1200 bp to be amplified. It is quite clear that only fragments of a small size (258 and 500 bp) can be amplified from degraded mitochondrial DNA, whereas the non-degraded DNA enables fragments of up to 1200 bp to be amplified.

FIGS. 7A and 7B show the amplification of nuclear DNA fragments (gene of RNA 18S, which is represented in the form of multiple copies in nuclear DNA) obtained from nuclear DNA extracted from fresh beef samples (non-degraded nuclear DNA; channels 2), or substrates containing degraded nuclear DNA taken from ravioli (channels 6). Channels 1 contain the size marker and channels 3, 4 and 5 represent the PCR carried out from the blank extraction in order to check for any contamination of the extraction reagents, amplification reagents or the environment.

In FIG. 7A, the following oligonucleotides were used:

| | |
|---|---|
| 18S1: | 5′ TAA CTG TGG TAA TTC TAG AG 3′ |
| 18S2: | 5′ ACT CTA GAT AAC CTC GGG CC 3′ |

These oligonucleotides enable a fragment of 163 bp to be amplified.

In FIG. 7B, the following oligonucleotides were used:

| | |
|---|---|
| 18S1: | 5′ TAA CTG TGG TAA TTC TAG AG 3′ |
| 18S3: | 5′ AGC TCC AAT AGC GTA TAT TA 3′ |

These oligonucleotides enable a fragment of 1200 bp to be amplified.

It is clear that, irrespective of the size of the amplified fragment, degraded nuclear DNA did not produce any positive amplification whereas the non-degraded nuclear DNA generated specific products for the two sizes of amplified fragments. This is very different from the results obtained with mitochondrial DNA.

FIG. 8 represents the amplification of nuclear DNA fragments (gene coding for foetal β-globin, which is represented in the form of unique copies in the nuclear DNA) obtained from nuclear DNA taken from samples of fresh beef (non-degraded nuclear DNA; channel 2), or from substrates containing degraded nuclear DNA taken from ravioli (channel 6). Channel 1 contains the size marker and channels 3, 4 and 5 represent the PCR obtained from the blank extraction taken in order to check for any contamination of the extraction reagents, amplification reagents or the environment. The following oligonucleotides were used:

| | |
|---|---|
| BGLO1: | 5′ GGA AGA GCT GGG CCA GCT GC 3′ |
| BGLO2: | 5′ CAG GTA GGT ATC CCA CTT AC 3′ |

These oligonucleotides enable a fragment of 180 bp to be amplified. It is clearly evident that the degraded nuclear DNA did not produce a positive amplification whereas the non-degraded nuclear DNA generated a specific product. This result is identical to that obtained on the nuclear gene with multiple copies, RNA 18S.

### EXAMPLES

Cloning may be used as a means of identifying animal and vegetable species present in a biological sample in different types of situations, the main ones of which are set out below:

CASE 1: the experimenter has no information about the biological product to be tested and simply wants to draw up a list of the species/populations/races/individuals present.

In this case, the mitochondrial DNA present is amplified using the most general primers possible, the product obtained from PCR is cloned and the largest possible number of clones is sequenced. Each different sequence therefore represents the sequence of a species, a population, a race or an individual. The greater the number of clones sequenced, the more complete the list of different species or populations or races or individuals present will be.

CASE 2: a priori, the experimenter has some idea of the type of species, populations, races or individuals present but wants to identify them more precisely.

For example, a food preparation is known to contain fish but there is no information about other species contained in it. In this case, the procedure is the same as for case 1 but the mitochondrial DNA is amplified with specific primers for fish.

CASE 3: the experimenter knows exactly what species or populations or races or individuals are present but wishes to confirm this.

It may then be of advantage to run a single amplification with generalist primers rather than run a series of independent amplifications with primers specific to each of the species. This might be the case if the sample were available in very small quantities, for example.

CASE 4: one species/population/race/individual contained in the product is known and it would be desirable to ascertain whether there is a mixture.

In this case, two generalist primers are used for amplification purposes, followed by cloning, and the sequence of each clone will make it possible to determine whether there are several species or populations or races or individuals, different or otherwise, in the preparation. It should be pointed out that the invention may be applied whether the nucleotide sequences of the amplified fragments of the species or populations or races or individuals are already known or not. Accordingly, if, for example, two sequences are found in a food preparation and cannot be found in the databases, this will mean that two distinct species or populations or races or individuals are present. By running a phylogenetic analysis, it will then be possible to determine the zoological group to which these different sequences belong. In the majority of cases, it will then be possible to identify the species, population, race or individual in question by analysing reference samples, unless the species or population or race or individual is one which is new to science. Even in the latter case, the invention would still make it possible to ascertain that several different species or populations or races or individuals are indeed present.

### Example 1

### Detection and Identification of a Mixture of at Least Three Species

Primers exist, which enable all the vertebrates (mammals, fish, birds . . . ) to be amplified. These primers were defined by Kocher (Kocher, T. D., Thomas, W. K., Meyer, A., Edwards, S. V., Paabo, S., Villablanca, F. X., and Wilson, A. C. 1989. Dynamics of mitochondrial DNA evolution in animals: amplification and sequencing with conserved primers. Proc. Natl. Acad. Sci. USA. 86: 6196-6200) and produce a an amplification fragment of 360 bp (SEQ ID No27) of cytochrome b of mitochondrial DNA. These primers are known as “transvertebrate primers”.

The sequences of the primers are as follows:

| | |
|---|---|
| 5′ - AAA CTG CAG CCC CTC AGA ATG ATA TTT GTC CTC A- 3′ | (SEQ ID No 1) |
| 5′ - AAA AAG CTT CCA TCC AAC ATC TCA GCA TGA TGA AA- 3′ | (SEQ ID No 2) |

Amplified sequence SEQ ID No27 conforms to the following formula:

TYCCHRCBCC VTCHAAYATY TCHKYHTGAT GRAAYTTYGG HTCHYTHYTD GSMVYNTGCY
TNATNMYHCA RMTYHYMACH GGHYTAYTHY TAGCHATRCA CTAYWCMBCN GACRYMDMVM
YMGCHTTYTC MTCHRTHRYC CAYAYYWSYC GDRAYGTDMA HTAYGGCTGA MTHATYCGVW
AYMTNCAYGC HAAYGGHGCH TCHWTVTTYT TYATYTGYWT HTWYMTDCAY RTHGSVCGMG
GHHTMTAYTA YGGNTCHTWY VYHTWYNHRG ARACMTGAAA YAYHGGVRTH RTMCTHYTVY
TYDYARYHAT ARYMACMKCH TTYTYRTRGG HTAYGTHCTM CCVTGRGGMC AAATATCATT

in which Y is C or T, H is A, C or T, R is A or G, V is A, C or G, K is G or T, D is A, G or T, S is C or G, M is A or C, N is A, C, G or T, W is A or T, B is C, G or T.

These primers are used if there is a suspected mixture of different animal groups, because this avoids having to run several amplifications with primer pairs capable of amplifying these different groups and hence having to run several cloning processes. For example, it would be possible to detect a mixture of species in a meat containing beef, pork, sheep meat, chicken, duck, . . .

In order to be able to set up a method for detecting and identifying a mixture of species by cloning, it is first necessary to extract DNA from biological materials and then run a gene amplification.

Extraction

1) Extraction of DNA by the Method Using Phenol/Chloroform

This method is used for all types of samples likely to contain organic matter, such as fillet, soup, terrine, pâté, fat, flour, fish-based preparations, etc.

This method is based on the techniques described in reference works by HÄNNI et al., 1990, C. R. Acad. Sci. Paris., 310, 365-370 and HÄNNI et al., 1995, Nucl. Acids Res., 23, 881-882, relating to the extraction of DNA from bones and teeth.

A quantity of approximately 1 to 2 g of a sample of organic matter is incubated for two hours at 37° C. in 400 μl of lysis buffer of the following composition:
STE 1×(NaCl 100 mM, Tris 10 mM at pH 7.4, EDTA (ethylene diamine tetracetic acid) 1 mM),
SDS 2%,
proteinase K at 0.5 mg/ml.

Proteinase K enables proteins to be degraded and nucleic acids to be released. The lysate is then extracted twice using a quantity of phenol/chloroform (1/1). The sample is centrifuged for 15 minutes at 1000 g and the organic phase removed so that the protein element of the lysate can be removed. The DNA is precipitated by a 1/10 volume of 2M sodium acetate and then by 2.5 volumes of isopropanol, and centrifuged for 30 minutes at 10,000 g. The DNA is absorbed in water: this results in a DNA extract. The volume of water will depend on the quantity of DNA recovered.

Migration of the DNA from various samples produces characteristic traces of a degraded DNA, although it can be amplified perfectly well.

2) Extracting DNA Using the Kit or Extraction Kit Method

This method is conducted under the conditions stipulated by the manufacturer, Qiagen.

Amplification:

The primer pair SEQ ID No2 and SEQ ID No8 as defined above are used for the PCR. The amplifications are conducted using a total volume of 50 μl containing:
200 μg/ml of BSA (Bovine Serum Albumin),
250 mM of dNTP (deoxynucleotide Triphosphate),
300 ng of each primer,
1.5 mM of magnesium chloride (MgCl₂),
PCR buffer 10×(100 mM Tris-HCl pH 8.3; 500 mM KCl),
1 unit of Taq Polymerase,
quantity of sterile distilled water to make up 50 μl,
1 μl of DNA extract.

The mixture is reacted in a sterile unit with a horizontal flow head to prevent contamination as far as possible. The PCR are conducted on PCR Ependorff apparatus. Each PCR is broken down as follows:
1 initial cycle at a temperature of 94° C. lasting 2 minutes followed by,
40 cycles at a temperature of 94° C. for 1 minute, at a temperature of 55° C. to 63° C. for 1 minute, at a temperature of 72° C. for 2 minutes; during the last cycle, a terminal elongation takes place at a temperature of 72° C. for 7 minutes.

The amplification products are analyzed by electrophoresis on 2% agarose gel at a constant voltage of 100 V for 30 min, using ethidium bromide in order to view the amplifications obtained.

The entire PCR amplification product (amplification product) can be purified directly using the “QIAquick PCR purification Kit” system sold by Qiagen, following the accompanying instructions. The entire PCR product is passed through a column of silica gel. The nucleic acids are retained on the column, whilst the other residues of the PCR are eluted to the micro-centrifugation unit. The impurities are removed and the DNA is eluted by Tris buffer or water. The purified DNA is then viewed and quantified on 2% agarose gel.

Direct Sequence of the Amplification Product

The purified PCR products are automatically sequenced.

The primers used are those used to amplify the DNA fragment or fragments to be characterized. The “Perkin-Elmer” kit is used for the PCR reaction, which is operated over 25 cycles under the conditions specified by the supplier. Each amplification product obtained from the PCR reaction is precipitated with ethanol and placed on a polyacrylamide gel. The resultant sequences are then compared with those of the databanks or with the sequences already obtained.

The DNA contained in a sample of organic matter containing a mixture of different species of vertebrates is extracted and amplified in the manner described above. The amplification product obtained from the PCR reaction is unique (containing at least one DNA fragment represented by a fragment with 360 base pairs) and is obtained using a primer pair (SEQ ID No1, SEQ ID No2) which enables all vertebrates to be amplified (see FIG. 3, well 2). Said amplification product is therefore likely to contain different DNA fragments characteristic of different species of vertebrates.

Cloning

The amplification product is cloned directly, with or without purification, using the “TOPO TA cloning kit” system sold by Invitrogen in accordance with the accompanying instructions, in order to isolate and obtain numerous identical copies of all the different DNA fragments contained in the amplification product. To this end, the amplification product is inserted in the plasmid “pCR®2.1-TOPO® 3.9 kb” sold by Invitrogen by ligation, (FIG. 1-1). Each DNA fragment contained in the amplification product will be inserted in a plasmid “pCR®2.1-TOPO®” by means of the DNA ligase enzyme (FIG. 1-2). A plasmid therefore corresponds to each DNA fragment. All the DNA fragments contained in the amplification product are then separated during this stage.

Once each of the DNA fragments have been ligated in a plasmid, each plasmid is introduced into a bacterium, for example Escherichia coli (E. coli) (FIG. 1-4), by a process known as “transformation” which involves applying an osmotic shock and a temperature shock or an electric shock. The E. coli bacteria thus obtained are cultivated on gelose so as to multiply in the presence of an antibiotic (for example ampicillin). The presence of the antibiotic enables the E. coli bacteria, incorporated in a plasmid, to be selected because plasmids naturally possess a gene which is resistant to an antibiotic. Accordingly, the E. coli bacteria which have not incorporated a plasmid will not be able to develop.

A visual system is used to select the E. coli bacteria which have incorporated a plasmid in which a DNA fragment has ligated. Actually, two reagents (IPTG and X-Gal) were introduced into the gelose-containing medium, which will produce a blue colouration in contact with the β-galactosidase enzyme. The colonies of blue bacteria obtained are the ones which synthesize β-galactosidase and which contain a plasmid in which a DNA fragment is not ligated. The colonies of white bacteria obtained are those which do not synthesize β-galactosidase and which contain a plasmid in which a DNA fragment is ligated. The latter is ligated due to the fact that the DNA fragment is inserted in the plasmid in the medium of a gene coding for the β-galactosidase enzyme, blocking its synthesis.

Each white bacterial colony contains a single amplified DNA fragment or a single amplified DNA sequence, said DNA fragment or said DNA sequence corresponding to a single and same species of gadiformes. The plasmid DNA containing the DNA fragments must then be recovered, i.e. separated from the bacterial DNA in readiness for sequencing.

In this example, the aim is to detect at least 3 species of vertebrates.

In one advantageous embodiment of the method proposed by the invention, about twenty plasmid DNA samples are recovered (denoted respectively by letters A, B, C, D, E, F, G, H, I, J, K, L, M, N, O, P, Q, R, S, and T in FIG. 1) from 20 independent white bacterial colonies selected at random.

The 20 plasmid DNA samples (denoted by letters A to T in FIG. 2) thus recovered are then sequenced with the primers included in the Invitrogen kit. The sequences obtained are analyzed in order to ascertain whether the profiles are different or not. The results obtained (FIG. 2) indicate that the sample of organic matter contains a mixture of four species, in this particular case Bos taurus (beef: fragments A, B, C, E, J and T), Ovis aries (sheep meat: fragments D, 1, K, 0, P and S), Sus scrofa (pork: fragments L, M and R) and Gallus gallus (chicken: fragments F, G, H, N, N and Q).

### Example 2

### Detecting and Identifying a Mixture of Species: Gadiformes

Certain food preparations can be prepared using several species of fish (ready-cooked meals, soup, pâtés, terrine . . . ). This mixture may be specified by the manufacturer but there may also be an adulteration. In practice, situations can arise in which this mixture contains more of a cheaper fish species than another species which ought to be the only one contained in the preparation (example: salt cod prepared with a small quantity of cod (Gadus morhua) and a large quantity of cheaper Pacific cod (Gadus macrocephalus).

If it is not known whether the sample contains several species of gadiformes, it is necessary to use the primer pairs that will enable all of them to be amplified.

The primer pair SEQ ID No3 and SEQ ID No4 results in fragment SEQ ID No5 with 442 bp.

SEQ ID No3 conforms to the following formula:

AYC ARC AYY TRT TYT GRT TCT

in which Y is C or T, R is A or G.

SEQ ID No4 conforms to the following formula:

TAY GTW GTN GCN CAY TTY CA

in which Y is C or T, W is A or T, N is A, C, G or T.

SEQ ID No5 conforms to the following formula:

AYCARCAYYT RTTCTGATTC TKCGGNCAYC CYGAAGTHTA YATCTNATY YTMCCHGGMT
TCGGRATAAT YTCYCAYATY GTAGCVTAYT AYTCAGGNAA RMAAGARCCN TTYGGRYAYA
TRGGHATRGT NTGAGCYATR ATRGCYATYG GMCTYCTYGG YTTTATYGTV TGRGCYCAYC
ACATRTTYAC AGTBGGRATR GAYGTDGAYA CMCGWGCHTA CTTYACATCY GCAACBATAA
TYATYGCYAT YCCRACAGGY GTWAAAGTYT TYAGYTGAYT AGCAACYYTV CAYGGRGGCT
CARTTAARTG RGAVACHCCB MTMCTBTGRG CCCTDGGYTT YATYTTYCTM TTYACMGTHG
GVGGMYTWAC AGGNATYRTH YTRGCYAAYT CYTCYCTAGA YATYGTDCTY CAYGAYACRT
AYTAMGTAGT MGCYCAYTTY CA

in which Y is C or T, R is A or G, K is G or T, N is A, C, G or T, H is A, C or T, M is A or C, V is A, C or G, B is C, G or T, D is A, G or T, W is A or T.

The primer pair SEQ ID No6 and SEQ ID No4 gives fragment SEQ ID No7 with 328 bp.

SEQ ID No6 conforms to the following formula:

GGN YAY ATR GGN ATR GTN TGA GC

in which N is A, C, G or T, Y is C or T, R is A or G.

SEQ ID No7 conforms to the following formula:

GGRYAYATRG GHATRGTNTG AGCYATRATR GCYATYGGMC TYCTYGGYTT TATYGTVTGR
GCYCAYCACA TRTTYACAGT BGGRATRGAY GTDGAYACMC GWGCHTACTT YACATCYGCA
ACBATAATYA TYGCYATYCC RACAGGYGTW AAAGTYTTYA GYTGAYTAGC ACYYTVCAYG
GRGGCTCART TAARTGRGAV ACHCCBMTMC TBTGRGCCCT DGGYTTYATY TTYCTMTTYA
CMGTHGGVGG MYTWACAGGN ATYRTHYTRG CYAAYTCYTC YCTAGAYATY GTDCTYCAYG
AYACRTAYTA MGTAGTMGCY CAYTTYCA

in which R is A or G, Y is C or T, K is G or T, N is A, C, G or T, H is A, C or T, M is A or C, V is A, C or G, B is C, G or T, D is A, G or T, W is A or T.

If the intention is to detect a mixture of gadides, the primers specified above may be used, which enable all gadiformes to be identified, or alternatively primers may be used which enable all the gadides to be amplified.

The primer pair SEQ ID No8 and SEQ ID No9 gives the fragment SEQ ID No10 with 386 bp.

SEQ ID No8 conforms to the following formula:

ACC AAC ACT TAT TCT GAT TCT

SEQ ID No9 conforms to the following formula:

GGC TTA ACA GGA ATT GTA CTA GCT

SEQ ID No10 conforms to the following formula:

AYCARCAYYT RTTCTGATTC TKCGGNCAYC CYGAAGTHTA YATHCTNATY YTMCCHGGMT
TCGGRATAAT YTCYCAYATY GTAGCVTAYT AYTCAGGNAA RMAAGARCCN TTYGGRYAYA
TRGGHATRGT NTGAGCYATR ATRGCYATYG GMCTYCTYGG YTTTATYGTV TGRGCYCAYC
ACATRTTYAC AGTBGGRATR GAYGTDGAYA CMCGWGCHTA CTTYACATCY GCAACBATAA
TYATYGCYAT YCCRACAGGY GTWAAAGTYT TYAGYTGAYT AGCAACYYTV CAYGGRGGCT
CARTTAARTG RGAVACHCCB MTMCTBTGRG CCCTDGGYTT YATYTTYCTM TTYACMGTHG
GVGGMYTWAC AGGNATYRTH YTRGCY

in which Y is C or T, R is A or G, K is G or T, N is A, C, G or T, H is A, C or T, M is A or C, V is A, C or G, B is C, G or T, D is A, G or T, W is A or T.

The primer pair SEQ ID No11 and SEQ ID No9 gives fragment SEQ ID No12 with 237 bp.

SEQ ID No11 conforms to the following formula:

GGC CTC CTT GGC TTT ATT GTA

SEQ ID No12 conforms to the following formula:

GGMCTYCTYG GYTTTATYGT VTGRGCYCAY CACATRTTYA CAGTBGGRAT RGAYGTDGAY
ACMCGWGCHT ACTTYACATC YGCAACBATA ATYATYGCYA TYCCRACAGG YGTWAAAGTY
TTYAGYTGAY TAGCAACYYT VCAYGGRGGC TCARTTAART GRGAVACHCC BMTMCTBTGR
GCCCTDGGYT TYATYTTYCT MTTYACMGTH GGVGGMYTWA CAGGNATYRT HYTRGCY

in which Y is C or T, R is A or G, K is G or T, N is A, C, G or T, H is A, C or T, M is A or C, V is A, C or G, B is C, G or T, D is A, G or T, W is A or T.

If the intention is to detect a mixture of merluccids, the primers specified above may be used which enable all the gadiformes to be identified, or alternatively primers which enable all the merluccids to be amplified.

The primer pair SEQ ID No13 and SEQ ID No14 gives fragment SEQ ID No15 with 318 bp.

SEQ ID No13 conforms to the following formula:

TAA TYT CYC AYA TYG TAG CC

in which Y is C or T.

SEQ ID No14 conforms to the following formula:

ACT TAC AGG NAT YRT HCT RG

in which N is A, C, G or T, Y is C or T, R is A or G, H is A, C or T.

SEQ ID No15 conforms to the following formula:

TAATYTCYCA YATYGTAGCV TAYTAYTCAG GNAARMAAGA RCCNTTYGGR YAYATRGGHA
TRGTNTGAGC YATRATRGCY ATYGGMCTYC TYGGYTTTAT YGTVTGRGCY CAYCACATRT
TYACAGTBGG RATRGAYGTD GAYACMCGWG CHTACTTYAC ATCYGCAACB ATAATYATYG
CYATYCCRAC AGGYGTWAAA GTYTTYAGYT GAYTAGCAAC YYTVCAYGGR GGCTCARTTA
ARTGRGAVAC HCCBMTMCTB TGRGCCCTDG GYTTYATYTT YCTMTTYACM GTHGGVGGMY
TWACAGGNAT YRTHYTRG

in which Y is C or T, R is A or G, K is G or T, N is A, C, G or T, H is A, C or T, M is A or C, V is A, C or G, B is C, G or T, Dis A, G or T, W is A or T.

The primer pair SEQ ID No16 and SEQ ID No14 gives fragment SEQ ID No17 with 189 bp.

SEQ ID No16 conforms to the following formula:

GRA TRG AYG TDG AYA CMC GT

in which R is A or G, Y is C or T, D is A, G or T, M is A or C.

SEQ ID No17 conforms to the following formula:

GRATRGAYGT DGAYACMCGW GCHTACTTYA CATCYGCAAC BATAATYATY GCYATYCCRA
CAGGYGTWAA AGTYTTYAGY TGAYTAGCAA CYYTVCAYGG RGGCTCARTT AARTGRGAVA
CHCCBMTMCT BTGRGCCCTD GGYTTYATYT TYCTMTTYAC MGTHGGVGGM YTWACAGGNA
TYRTHYTRG

in which R is A or G, Y is C or T, D is A, G or T, M is A or C, W is A or T, B is C, G or T, V is A, C or G, H is A, C or T, N is A, C, G or T.

Several examples of the mixtures of species which can be identified using this system are set out below.

The first is a mixture containing gadiformes for example of the Alaska pollack (Theragra chalcogramma), which is a gadide, and Argentine hake (Merluccius hubbsi) which is a merluccid. The primer pair (SEQ ID No6 and SEQ ID No4), which enables all gadiformes to be amplified, and gives a fragment (SEQ ID No7) with 328 bp, is used. Two sequence profiles are observed on the ten clones analyzed. When compared with reference sequences grouped in a bank of sequences (Genebank for example), these two profiles correspond to a species of gadide: the Alaska pollack (Theragra chalcogramma) and a species of merluccid: Argentine hake (Merluccius hubbsi).

The second is a mixture between gadides of the Atlantic cod for example (Gadus morhua) and ling (Molva molva). The primer pair (SEQ ID No11 and SEQ ID No9), which enables all gadides to be amplified and produces a fragment (SEQ ID No12) with 237 bp, is used. This fragment is cloned and then sequenced. Two sequence profiles may be observed on the ten clones analyzed. When compared with reference sequences grouped in a bank of sequences (Genebank for example), these two profiles correspond to two species of gadides: Atlantic cod (Gadus morhua) and ling (Molva molva).

The third type of mixture is a mixture containing merluccids, for example Cape hake (Merluccius capensis) and common hake (Merluccius merluccius). The primer pair (SEQ ID No 16 and SEQ ID No14), which enables all the merluccids to be identified and gives a fragment (SEQ ID No17) with 189 bp, is used. This fragment is cloned and then sequenced. Two sequence profiles are observed on the ten clones analyzed. When compared with reference sequences in a bank of sequences (Genebank for example), these two profiles correspond to two species of merluccids: Cape hake (Merluccius capensis) and common hake (Merluccius merluccius).

### Example 3

### Detecting and Identifying a Mixture of Species: Anseriformes

In the case of anseriformes, different forms of mixtures may exist. The first relates to products such as foie gras, wherein goose can be largely replaced by duck, which is cheaper, and foie gras of duck can also be substituted by hen in varying proportions. These mixtures of species in foie gras are primarily to be found in blocks of foie gras, which are livers reconstituted from several livers and not whole livers from a single individual. Other types of mixtures are found in ready-cooked meals, prepared from different species.

Mixtures of anseriforme species are detected by cloning the fragment obtained by the primer pair defined by the patent for detecting anseriformes. This primer pair (SEQ ID No18 and SEQ ID No19) gives a fragment (SEQ ID No20) with 203 bp. All the anseriforme species can therefore be detected in whatever mixture.

SEQ ID No18 conforms to the following formula:

ACT AGC TTC AGG CCC ATA CG

SEQ ID No19 conforms to the following formula:

AAA AAT AAA AGG AAC CAG AG

SEQ ID No20 conforms to the following formula:

ACYAGCTTCA GGCCCATACG TTCCCCCTAA ACCCCTCGCC CTCCTCACAT TTTTGCGCCT
CTGGTTCCTC GGTCAGGGCC ATCMATTGGG TTCACTCACC YCYMYTYGCC YTTCAAAGTG
GCATCTGTGG ANKACBTYCA CCWYYYCRRT GCGTWATCGC GGCATBYTYM ASYWTTTWSM
CGCCTYTGGT TCYMYTTHTY TYT

in which Y is C or T, M is A or C, N is A, C, G or T, K is G or T, B is C, G or T, W is A or T, R is A or G, S is C or G.

### Example 4

### Detecting and Identifying a Mixture of Species: Salmon

Cases of fraud exist, based on the single species of Atlantic salmon Salmo salar and the five species of Pacific salmon of the genus Oncorhynchus. In Europe, two species are bred on an industrial scale, Atlantic salmon (Salmo salar) and rainbow trout (Oncorhynchus mykiss). These species are generally smoked and once smoked it is very difficult to make a distinction between these two species, both from a visual point of view and on the basis of their texture by touch. These two species therefore do not sell at the same price because salmon is much more expensive than trout. A lot of fraud therefore goes on involving this product, in cases where salmon is not sold in the form of fillets but in another type of format, especially during the Christmas and New Year holiday periods.

Several primer pairs have been defined as a means of amplifying all salmonides. Two molecular markers have been selected.

For the region controlling replication of mtDNA, the primer pair:

SEQ ID No21 conforming to the following formula:

GCC GAA TGT AAA GCA TCT GG

SEQ ID No22 conforming to the following formula:

ACC TTA TGC ACT TGA TAT CC

gives a fragment of 245 bp (SEQ ID No23)

SEQ ID No23 conforms to the following formula:

GCCBWWHDWR VVRCAYSTKB BBMWTRVWKH MRATCWYRWT GCSCGTTRMT CRCVRMRYCK
DBCRBYYTHT TRWVYRCHYM BGGKWSYYYT YHWTKYWTYY TTWYCKYHYR GSKKGYMYDY
MCMRRTRSMA BYDMKRKRSK CYVRMRMBSK MRVMCYVKAY STYGMATTCC AGAGARYMYM
TGYVTYWKVK YSMWRYSHYA THCTMTHADK DATYRCWYMH TKRGAYRKYH RARYATAHRG
KBRAT

in which B is C, G or T, W is A or T, H is A, C or T, D is A, G or T, R is A or G, V is A, C or G, Y is C or T, S is C or G, K is G or T, M is A or C.

In the case of the cytochrome b of mtDNA, the primer pair:

SEQ ID No24 conforming to the following formula:

ACC GGG TCT AAT AAC CCA GC

SEQ ID No25 conforming to the following formula:

ATG ATA ATG AAT GGG TGT TC

gives a fragment of 442 bp (SEQ ID No26)

SEQ ID No26 conforms to the following formula:

WCVGGVTCHA AYAACCCMVY AGGHATYWMM TCMSAYKYHG AYAAAATYHC MTTYCACCCH
TACTWYWCMW WYAARGACVY YYTNGGMTTN VYHSYYWTMC THMYYKBHMT RAYAYYMYTA
RYHCTRTTCK CMCCMRACCT CCTMGGVGAC CCRGAHAAYT WYACVCYWGC MAAYCCMYTM
RWHACHCCHC CHCAWATCAA RCCHGARTGA TAYTTYYTAT TYGCMTAYRC MATYYTHCGM
TCMRTYCYAA YAAACTWGGM GGHGTMCTHG CCCTMKYMBY MTCNRTCCTV RTYCTHDYHV
TMRTYCCYHT MCTMCAYAHM TCYAARCAAC RMRSMMTRAY MTTYCGMCCM CTMWSCCAAW
BMYTWTWYTG RVYYCTRGYM GCVRACMTHC TNAYHCTHAC MTGAATYGGR RGVMWACCHG
TVRRMYACCC HTWYAYYAYC ATYGG

in which W is A or T, V is A, C or G, H is A, C or T, Y is C or T, M is A or C, S is C or G, K is G or T, R is A or G, N is A, C, G or T, D is A, G or T, B is C, G or T.

The primer pair defined on the basis of the control region is chosen by preference because there is a better chance of obtaining it in the case of degraded foodstuffs due to its smaller size.

### Example 5

### Detecting and Identifying a Mixture of Individuals

This type of detection may also be used for a single species which is made up of several individuals. This is the case with the human species, for which it has been demonstrated that the region controlling mtDNA enabled a distinction to be made between individuals based on maternal lineage. This observation is very widely used in laboratories working with genetic fingerprinting for specifying individual DNA types.

For example, if several examples of human DNA are found on a sample under analysis, the cloning method will enable the individual samples of human DNA found to be sorted and each of the individuals identified independently on the basis of its DNA.

Such a situation might occur where organic traces from several individuals are mixed (for example 1f several persons have drunk from the same glass, thus leaving several types of different traces behind). If the intention is to use mitochondrial DNA as a means of identification in such a situation, a sequence is obtained containing a number of indeterminate samples, which makes identification impossible. Cloning gets round this problem by separating each sequence, thereby enabling several distinct and specific signatures to be obtained.

Cloning also makes it possible to detect the presence of several individuals, whereas direct analysis of the sequence would not permit this. For example, if old traces of organic samples are found (a spot of old blood several years old, for example) and these samples have been mixed with recent organic samples (capillary tissue of any person more recently in contact), a DNA extract containing an old, very degraded DNA sample and a modern DNA sample in good condition will be obtained. In such a case, the modern DNA sample is preferably amplified and will be very much in the majority, so that a single signature can be obtained which is visible in the direct sequence. If cloning is applied, however, and a large number of clones is analyzed (for example 30), it will be possible to obtain a majority (for example 28) of contaminating clones from the modern DNA sample and a minority (for example 2) from the degraded DNA sample. Cloning therefore discloses the presence of a mixture, whereas none of the conventional analysis methods could have predicted this. Consequently, this method enables a much more refined analysis to be conducted on organic samples, detecting and/or revealing the presence of unsuspected sequences corresponding to individuals.

In the example described, bone fragments were extracted by the methods conventionally used in molecular archaeology (Anderson et al. “Sequence and organization of the human mitochondrial genome” Nature 1981 vol 290, 457-465). A region of 382 bp from the region controlling mitochondrial DNA was amplified with the following oligonucleotides:

| | |
|---|---|
| AAG CAG ATT TGG GTA CCA C-3′ | (SEQ ID N^{o} 31) |
| GAT TTC ACG GAG GAT GGT G-3′ | (SEQ ID N^{o} 32) |

The amplified region corresponds to positions 16037 (SEQ ID No31) to 16419 (SEQ ID No32) of human mitochondrial DNA as specified by Anderson in 1981 (Nature 1981).

This region is compared with the 3 sequences obtained by cloning this fragment (respectively SEQ ID No33, SEQ ID No 34, and SEQ ID No35) and with the direct sequence of the fragment obtained from PCR (DIRECT). It is evident that a letter N in the direct sequence coincides with each difference between one of the 3 sequences. From this, it was concluded that the bone fragments contain genetic material from 3 different individuals.

### Example 6

### Detecting and Identifying a Mixture of Vertebrates in a can of Pet Food

The stages are the same as those defined in example 1: extraction, amplification with the transvertebrate primer pair, cloning, sequencing. What is different is the analysis of the results because in this example, in addition to having sequenced the amplification product obtained using the transvertebrate primer pair (FIG. 3, well 2), the results are also confirmed with the aid of specific primers for the species of vertebrates, which are found after sequencing the plasmid DNA.

The “transvertebrate” primers are therefore used to amplify a fragment of cytochrome b of the mitochondrial DNA of a sample taken from a can of pet food.

In this example, the aim is to find a mixture in which the number of species is unknown. The label states the contents as being “minced chicken and duck in gelatine”, followed by a list of ingredients (meats and animal sub-products). It is therefore assumed that there will certainly be more than two species in addition to chicken and duck, such as pork and beef.

In one advantageous embodiment of the method proposed by the invention, about thirty plasmid DNA samples are recovered (denoted respectively by letters A, B, C, D, E, F, G, H, I, J, K, L, M, N, O, P, Q, R, S, T, U, V, W, X, Y, Z, AA, AB, AC, AD, AE in FIG. 3-b) taken from 30 independent white bacterial colonies selected at random.

The 30 plasmid DNA samples (denoted by letters A to AE in FIG. 3-b) thus recovered are then sequenced using the primers supplied with the Invitrogen kit. The sequences obtained are analyzed in order to ascertain whether there are different profiles or not. The obtained results (FIG. 3-b) indicate that the sample of organic matter contains a mixture of seven species, in this particular case Bos taurus (beef: fragments A, F, H, K, 0, Q and V), Sus scrofa (pork: fragments C, I, J and M)), Gallus gallus (chicken: fragments B, D, P, R, S, U and X), Cairina moschata (Barbary duck: fragments E, G, L, AA and AD), Salmo trutta (salmon: fragments: T, Z and AE) and Theragra chalcogramma (pollack: N, Y, AB and AC).

### Example 7

### Detecting and Identifying a Vegetable Mixture

A primer pair has been defined on the chloroplast genome as a means of amplifying the gene coding for the sub-unit of ribulose 1,5-bisphosphate carboxylase/oxygenase (rbcL), which is contained in all vegetable matter. The resultant amplification product enables different vegetables species to be detected and identified.

One example relates to a system of detecting and identifying cepages because wine generally contains several cepages in its composition. The cepages, for example cabernet franc, chardonnay, chasselas, gamay, sauvignon or syrah, all belong to a same species (Vitis vinifera). The primer sequences are as follows:

| | |
|---|---|
| AAA GCT CTG CGC GCT CTA CG | (SEQ ID No 28) |
| CCG CGG AGA CAT TCA TAA AC | (SEQ ID No 29) |

SEQ ID No28 and SEQ ID No29 give a fragment of 202 bp (SEQ ID No30)

SEQ ID 30 conforms to the following formula:

AAAGCTCTGC GCGCTCTACG TCTARAGGAT CTGCGAATCC CCCTGCTTAT ACTAAAACTT
TCCAAGGCCC GCCTCATGGC ATCCAAGTTG AGAGAGATAA ATTGAACAAG TATGGTCGTC
CCCTATTGGG ATGTACTATT AAACCTAAAT TGGGGTTATC CGCTAAGAAC TATGGTAGAG
CWGTTTATGA ATGTCTCCGC GG

in which R is A or G, W is A or T.

In order to develop a method for detecting and identifying a cepage mixture by cloning, it is necessary first of all to extract DNA from a biological material, followed by gene amplification.

Extraction with CTAB (cetyltrimethyl ammonium bromide).

This method, described by MURRAY (Nucleic Acid Res. 8 (19), 4321-4325 (1980)), is used by preference for vegetable samples.

Ig of sample is incubated for 3 hours at 56° C. in 2.5 ml of lysis buffer of the following composition:
Tris 10 mM pH8
EDTA 0.1 mM
sodium dodecylsulphate 1%
proteinase K 100 μg/ml
450 μl of NaCl 5 M and 375 μl of CTAB 10% NaCl 0.7 M, preheated to 65° C., are added and the mixture incubated for 20 min at 65° C. A quantity of chloroform is then added. After agitation and centrifugation (10 min at 12000 rpm at 4° C.), the aqueous phase is collected and re-extracted a second time to clarify it. It is then extracted again with a quantity of phenol/chloroform. The procedure after extraction is the same as that outlined in paragraph 1.

Amplification: (see example 1 above)

Cloning (see example 1 above)

### Example 8

### Experiments Comparing Degraded Mitochondrial DNA and Degraded Nuclear DNA

If a comparison is made on mitochondrial DNA between the capacity for amplification of fragments of different sizes, it becomes very evident that if the non-degraded mitochondrial DNA from fresh substrate enables fragments of both small and large size to be amplified (up to 1200 bp), whilst degraded mitochondrial DNA from processed substrates (such as in the example of ravioli containing pure beef) enables amplification of small fragments only (in this case 258 bp), whereas fragments of 500 and 1200 bp cannot be amplified. It is important to note that amplification of the small fragment in degraded DNA provides clear proof that, even though it would be impossible to see the DNA by direct visualisation after extraction (see FIG. 5, canal 4), an amplification product can be detected after PCR (see FIG. 6A, canal 6). The various checks for contamination which are conducted prove that the amplification product obtained is not caused by contamination of the samples by exogenic DNA.

By contrast with the results obtained with mitochondrial DNA, the DNA amplifications conducted on nuclear DNA do not allow amplification to be obtained if degraded nuclear DNA is used as a substrate. Although small mitochondrial fragments would enable an amplification to be obtained with degraded nuclear DNA, this is not so in the case of the nuclear gene 18S, irrespective of the size of the fragment in question (FIGS. 7A and 7B). This clearly demonstrates that amplifying small mitochondrial DNA fragments enables work to be conducted on a whole range of substrates which is not possible through amplification if looking for nuclear genes. Similar results were obtained on a nuclear gene with a unique copy, the β-globin foetal gene (FIG. 8). Here again, DNA amplification is only possible if working with a non-degraded substrate.

1. A cloning method for determining the existence of a mixture of organic origin containing mitochondrial or chloroplast DNA of different animal species, populations or races and/or different vegetable species, populations or races and/or different human individuals, comprising: amplifying each DNA sequence present, cloning the amplification product to separate different DNA sequences present in the mixture, and sequencing each DNA sequence.

2. The method according to claim 1, characterized in that the mitochondrial or chloroplast DNA is degraded.

3. The method according to claim 1 to detect and/or identify the DNA of each of the different animal species, populations or races and/or each of the different vegetable species, populations or races and/or each of the different human individuals present in a mixture of organic origin containing DNA from animal species, populations or races and/or from vegetable species, populations or races and/or from human individuals.

4. The method according to claim 1, in which each animal species, population or race and/or each vegetable species, population or race and/or each human individual is represented by at least one DNA sequence or at least one DNA fragment, and in particular by a unique DNA sequence or a unique DNA fragment, each of said DNA sequences or each of said DNA fragments being taken from DNA extracted from the mixture of organic origin.

5. The method according to claim 1 to detect and/or identify the DNA of each of the different animal species present in a mixture of animal species and in particular the DNA of each of the sub-species, lineages, races, varieties and/or strains and/or populations present in said mixture.

6. The method according to claim 1 for detecting and/or identifying the DNA of each of the different vegetable species present in a mixture of vegetable species and in particular the DNA of each of the sub-species, lineages, races, varieties and/or strains, cepage and/or populations present in said mixture, each of said vegetable species, and in particular each of said sub-species, races, varieties and/or strains, being represented by at least one DNA sequence or at least one DNA fragment, and in particular by a unique DNA sequence or a unique DNA fragment.

7. The method according to claim 1 for detecting and/or identifying the DNA of each of the different human individuals present in a mixture of DNA from different human individuals, each of said human individuals being represented by at least one DNA sequence or at least one DNA fragment, and in particular by a unique DNA sequence or a unique DNA fragment.

8. The method according to claim 1, in which the DNA extracted from the mixture of organic origin is old (or fossil) DNA, degraded DNA or modern DNA.

9. The method according to claim 1, in which the DNA extracted from the mixture of organic origin is:
non-degraded DNA, in particular taken from a fresh organic sample or,
degraded DNA, in particular taken from a processed sample, in particular one which has been cooked, freeze-dried, dried, preserved in brine, canned or pasteurized.

10. The method according to claim 1 for detecting and possibly identifying the DNA of each of the different animal species, populations or races and/or each of the different vegetable species, populations or races and/or each of the different human individuals present in the mixture of organic origin, in which at least one of said species, populations or races and/or at least one of said individuals is represented by an old DNA sequence or an old DNA fragment.

11. The method according to claim 1, in which the cloning method is preceded by a stage during which each of the DNA sequences or each of the DNA fragments characteristic of each of the animal species, populations or races and/or each of the vegetable species, populations or races and/or each of the human individuals to be detected and/or identified is amplified, the amplified DNA sequences or the amplified DNA fragments obtained from the amplification process being contained in a unique amplification product.

12. The method according to claim 11, in which each amplified DNA sequence or each amplified DNA fragment is of nuclear or mitochondrial origin if seeking to detect and/or identify a mixture containing DNA from different animal species and/or different human individuals and is of nuclear, mitochondrial or chloroplastal origin if seeking to detect and/or identify a mixture containing DNA from different vegetable species.

13. Method for detecting and/or identifying the DNA of each of the different animal species, populations or races and/or each of the different vegetable species, populations or races and/or of each of the different human individuals present in a mixture of organic origin, each of said animal species, population or race and/or each of said vegetable species, population or race and/or each of said human individuals being represented by at least one DNA sequence or at least one DNA fragment, in particular a unique DNA sequence or a unique DNA fragment, each of said DNA sequences or each of said DNA fragments being taken from the DNA extracted from said mixture, said method being characterized in that it comprises the following stages:
amplification of each of the DNA sequences or each of the DNA fragments characteristic of the animal species, population or race, of the vegetable species, population or race, or of the human individual to be detected and/or identified, in particular by the polymerase chain amplification method (PCR), in order to obtain a unique amplification product containing the different amplified DNA fragments or the different amplified DNA sequences,
cloning the amplification product obtained from the preceding amplification stage, in order to separate the different DNA sequences or the different DNA fragments present in the mixture of organic origin,
sequencing each of the DNA sequences or each of the DNA fragments thus separated from said mixture.

14. Method of detection and/or identification according to claim 13, in which:
the polymer chain amplification method (PCR) includes repeating the cycle of the following stages:
heating of the DNA extracted from the mixture of organic origin in order to separate the DNA into two single-chain strands,
hybridization of the single-chain DNA strands at an appropriate temperature with the appropriate oligonucleotide primers in order to amplify the DNA of the animal or vegetable species, populations or races or the human individuals to be detected and/or identified,
elongation of said appropriate oligonucleotide primers with a polymerase at an adequate temperature in order to obtain a unique amplification product containing each of said DNA sequences or each of said DNA fragments characteristic of a given animal or vegetable species or a human individual,
cloning the amplification product obtained from the preceding amplification stage, comprising the following stages:
with a DNA ligase enzyme, inserting said unique amplification product containing the different amplified DNA sequences or the different amplified DNA fragments characteristic of the DNA of the animal species, population or race, the vegetable species, population or race or the human individual to be detected and/or identified, in a previously linearized plasmid, in order to obtain several plasmids, each containing a unique amplified DNA sequence or a unique amplified DNA fragment,
incorporating each plasmid obtained from the preceding stage respectively in a bacterium, in particular Escherichia coli (E. coli),
multiplying each of the bacteria obtained in the preceding stage by cultivating said bacteria in an appropriate medium, in the presence of an antibiotic, in particular ampicillin, in order to select the bacteria which have incorporated a plasmid in which a DNA fragment or a DNA sequence has been inserted,
separating each of the plasmids containing a unique DNA sequence or a unique DNA fragment from each of the bacteria containing said plasmids in order to recover all the plasmids or “plasmid DNA” thus multiplied, each of said plasmids containing a unique DNA sequence or a unique DNA fragment,
taking a sufficient number of plasmids samples (or “plasmid DNA”) from all the plasmids (or “plasmid DNA”) obtained from the preceding stage in order to detect and/or identify at least two different DNA fragments or DNA sequences, each of said DNA sequences or DNA fragments being characteristic of a given animal or vegetable species, population or race, or of a human individual.

15. Method of detection and/or identification according to claim 14, in which each of the plasmid DNA samples obtained after the cloning stage is identified by sequencing, which enables the DNA characteristic of each of the animal species, populations or races and/or each of the vegetable species, populations or races and/or each of the human individuals present in the mixture of organic origin to be identified.

16. Oligonucleotides characterized in that they are selected from those:
1) presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No21 (position 16123 to 16144 of the mitochondrial DNA of salmon—replication control region according to Hurst, et al., 1999. Gene 239: 237-242):
GCC GAA TGT AAA GCA TCT GG
2) or those presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No22 (position 16341 to 16361 of the mitochondrial DNA of the salmon—replication control region—according to Hurst, et al., 1999. Gene 239: 237-242):
ACC TTA TGC ACT TGA TAT CC
3) or those presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No24 (position 14985 to 14996 of the mitochondrial of the salmon—cytochrome b—according to Hurst, et al., 1999. Gene 239: 237-242):
ACC GGG TCT AAT AAC CCA GC
4) or those presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No25 (position 15409 to 15430 of the mitochondrial DNA of the salmon—cytochrome b—according to Hurst, et al., 1999. Gene 239: 237-242):
ATG ATA ATG AAT GGG TGT TC
5) or those presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No28 (position 356 to 375 of the mitochondrial DNA of the salmon—replication control region—according to Albert et al., 1992, Science 257 (5076), 1491-1495):
AAA GCT CTG CGC GCT CTA CG
6) or those presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No29 (position 539 to 558 of the mitochondrial DNA of the salmon—replication control region—according to Albert et al., 1992, Science 257 (5076), 1491-1495):
CCG CGG AGA CAT TCA TAA AC

17. Primer pairs characterized in that they are constituted by:
the oligonucleotides SEQ ID No21 and SEQ ID No22
the oligonucleotides SEQ ID No24 and SEQ ID No25
the oligonucleotides SEQ ID No28 and SEQ ID No29

18. DNA fragment as amplified using a primer pair according to claim 17 and containing approximately 100 to approximately 500 base pairs.

19. DNA fragment according to claim 18, characterized in that it presents a sequence identity of at least 80%, preferably 90% and advantageously 95% with at least one of the sequences contained in:
SEQ ID No23 below:
GCCBWWHDWR VVRCAYSTKB BBMWTRVWKH
MRATCWYRWT GCSCGTTRMT CRCVRMRVCK
DBCRBYYTHTTRWVYRCHYM BGGKWSYYYT YHWTKYWTYY
TTWYCKYHYR GSKKGYMYDY
MCMRRTRSMABYDMRRRRSK CYVRMRMBSK MRVMCYVKAY
STYGMATTCC AGAGARYMYM
TGYVTYWKVKYSMWRYSHYA THCTMTHADK DATYRCWYMH
TKRGAYRKYH RARYATAHRG
KBRAT
in which B is C, G or T, W is A or T, H is A, C or T, D is A, G or T, R is A or G, V is A, C or G, Y is C or T, S is C or G, K is G or T, M is A or C,
or SEQ ID No26 below:
WCVGGVTCHAAYAACCCMVY AGGHATYWMM TCMSAYKYHG
AYAAAATYHC MTTYCACCCH
TACTWYWCMWWYAARGACVY YYTNGGMTTN VYHSYYWTMC
THMYYKBHMT RAYAYYMYTA
RYHCTRTTCKCMCCMRACCT CCTMGGVGAC CCRGAHAAYT
WYACVCYWGC MAAYCCMYTM
RWHACHCCHCCHCAWATCAA RCCHGARTGA TAYTTYYTAT
TYGCMTAYRC MATYYTHCGM
TCMRTYCYAAYAAACTWGGM GGHGTMCTHG CCCTMKYMBY
MTCNRTCCTV RTYCTHDYHV
TMRTYCCYHTMCTMCAYAHM TCYAARCAAC RMRSMMTRAY
MTTYCGMCCM CTMWSCCAAW
BMYTWTWYTGRVYYCTRGYM GCVRACMTHC TNAYHCTHAC
MTGAATYGGR RGVMWACCHG
TVRRMYACCCHTWYAYYAYC ATYGG
in which W is A or T, V is A, C or G, H is A, C or T, Y is C or T, M is A or C, S is C or G, K is G or T, R is A or G, N is A, C, G or T, D is A, G or T, B is C, G or T,
or SEQ ID No30 below:
AAAGCTCTGCGCGCTCTACG TCTARAGGAT CTGCGAATCC
CCCTGCTTAT ACTAAAACTT
TCCAAGGCCCGCCTCATGGC ATCCAAGTTG AGAGAGATAA
ATTGAACAAG TATGGTCGTC
CCCTATTGGGATGTACTATT AAACCTAAAT TGGGGTTATC
CGCTAAGAAC TATGGTAGAG
CWGTTTATGAATGTCTCCGC GG
in which R is A or G, W is A or T.


SEQUENCE LISTING (104 sequences)

SEQ ID NO: 1 - DNA, 34 bp, Artificial Sequence
  Description of the artificial sequence transvertebrate PCR primer
aaactgcagcccctcagaatgatatttgtcctca

SEQ ID NO: 2 - DNA, 35 bp, Artificial Sequence
  Description of the artificial sequence transvertebrate PCR primer
aaaaagcttccatccaacatctcagcatgatgaaa

SEQ ID NO: 3 - DNA, 21 bp, Artificial Sequence
  Description of the artificial sequence PCR primer
aycarcayytrttytgrttct

SEQ ID NO: 4 - DNA, 20 bp, Artificial Sequence
  Description of the artificial sequence PCR primer
taygtwgtngcncayttyca

SEQ ID NO: 5 - DNA, 442 bp, Artificial Sequence
  Description of the artificial sequence DNA fragment amplified from gadiformes genome
aycarcayytrttctgattctkcggncayccygaagthtayathctnatyytmcchggmttcggrataatytcycayatygtagcvtaytaytcaggnaarmaagarccnttyggryayatrgghatrgtntgagcyatratrgcyatyggmctyctyggytttatygtvtgrgcycaycacatrttyacagtbggratrgaygtdgayacmcgwgchtacttyacatcygcaacbataatyatygcyatyccracaggygtwaaagtyttyagytgaytagcaacyytvcayggrggctcarttaartgrgavachccbmtmctbtgrgccctdggyttyatyttyctmttyacmgthggvggmytwacaggnatyrthytrgcyaaytcytcyctagayatygtdctycaygayacrtaytamgtagtmgcycayttyca

SEQ ID NO: 6 - DNA, 23 bp, Artificial Sequence
  Description of the artificial sequence PCR primer
ggnyayatrggnatrgtntgagc

SEQ ID NO: 7 - DNA, 328 bp, Artificial Sequence
  Description of the artificial sequence DNA fragment amplified from gadiformes genome
ggryayatrgghatrgtntgagcyatratrgcyatyggmctyctyggytttatygtvtgrgcycaycacatrttyacagtbggratrgaygtdgayacmcgwgchtacttyacatcygcaacbataatyatygcyatyccracaggygtwaaagtyttyagytgaytagcacyytvcayggrggctcarttaartgrgavachccbmtmctbtgrgccctdggyttyatyttyctmttyacmgthggvggmytwacaggnatyrthytrgcyaaytcytcyctagayatygtdctycaygayacrtaytamgtagtmgcycayttyca

SEQ ID NO: 8 - DNA, 21 bp, Artificial Sequence
  Description of the artificial sequence PCR primer
accaacacttattctgattct

SEQ ID NO: 9 - DNA, 24 bp, Artificial Sequence
  Description of the artificial sequence PCR primer
ggcttaacaggaattgtactagct

SEQ ID NO: 10 - DNA, 386 bp, Artificial Sequence
  Description of the artificial sequence DNA fragment amplified from gadiformes genome
aycarcayytrttctgattctkcggncayccygaagthtayathctnatyytmcchggmttcggrataatytcycayatygtagcvtaytaytcaggnaarmaagarccnttyggryayatrgghatrgtntgagcyatratrgcyatyggmctyctyggytttatygtvtgrgcycaycacatrttyacagtbggratrgaygtdgayacmcgwgchtacttyacatcygcaacbataatyatygcyatyccracaggygtwaaagtyttyagytgaytagcaacyytvcayggrggctcarttaartgrgavachccbmtmctbtgrgccctdggyttyatyttyctmttyacmgthggvggmytwacaggnatyrthytrgcy

SEQ ID NO: 11 - DNA, 21 bp, Artificial Sequence
  Description of the artificial sequence PCR primer
ggcctccttggctttattgta

SEQ ID NO: 12 - DNA, 237 bp, Artificial Sequence
  Description of the artificial sequence PCR primer
ggmctyctyggytttatygtvtgrgcycaycacatrttyacagtbggratrgaygtdgayacmcgwgchtacttyacatcygcaacbataatyatygcyatyccracaggygtwaaagtyttyagytgaytagcaacyytvcayggrggctcarttaartgrgavachccbmtmctbtgrgccctdggyttyatyttyctmttyacmgthggvggmytwacaggnatyrthytrgcy

SEQ ID NO: 13 - DNA, 20 bp, Artificial Sequence
  Description of the artificial sequence PCR primer
taatytcycayatygtagcc

SEQ ID NO: 14 - DNA, 20 bp, Artificial Sequence
  Description of the artificial sequence PCR primer
acttacaggnatyrthctrg

SEQ ID NO: 15 - DNA, 318 bp, Artificial Sequence
  Description of the artificial sequence DNA fragment amplified from gadiformes genome
taatytcycayatygtagcvtaytaytcaggnaarmaagarccnttyggryayatrgghatrgtntgagcyatratrgcyatyggmctyctyggytttatygtvtgrgcycaycacatrttyacagtbggratrgaygtdgayacmcgwgchtacttyacatcygcaacbataatyatygcyatyccracaggygtwaaagtyttyagytgaytagcaacyytvcayggrggctcarttaartgrgavachccbmtmctbtgrgccctdggyttyatyttyctmttyacmgthggvggmytwacaggnatyrthytrg

SEQ ID NO: 16 - DNA, 20 bp, Artificial Sequence
  Description of the artificial sequence PCR primer
gratrgaygtdgayacmcgt

SEQ ID NO: 17 - DNA, 189 bp, Artificial Sequence
  Description of the artificial sequence DNA fragment amplified from gadiformes genome
gratrgaygtdgayacmcgwgchtacttyacatcygcaacbataatyatygcyatyccracaggygtwaaagtyttyagytgaytagcaacyytvcayggrggctcarttaartgrgavachccbmtmctbtgrgccctdggyttyatyttyctmttyacmgthggvggmytwacaggnatyrthytrg

SEQ ID NO: 18 - DNA, 20 bp, Artificial Sequence
  Description of the artificial sequence PCR primer
actagcttcaggcccatacg

SEQ ID NO: 19 - DNA, 20 bp, Artificial Sequence
  Description of the artificial sequence PCR primer
aaaaataaaaggaaccagag

SEQ ID NO: 20 - DNA, 203 bp, Artificial Sequence
  Description of the artificial sequence DNA fragment amplified from anseriformes genome
acyagcttcaggcccatacgttccccctaaacccctcgccctcctcacatttttgcgcctctggttcctcggtcagggccatcmattgggttcactcaccycymytygccyttcaaagtggcatctgtggankacbtycaccwyyycrrtgcgtwatcgcggcatbytymasywtttwsmcgcctytggttcymytthtytyt

SEQ ID NO: 21 - DNA, 20 bp, Artificial Sequence
  Description of the artificial sequence PCR primer
gccgaatgtaaagcatctgg

SEQ ID NO: 22 - DNA, 20 bp, Artificial Sequence
  Description of the artificial sequence PCR primer
accttatgcacttgatatcc

SEQ ID NO: 23 - DNA, 245 bp, Artificial Sequence
  Description of the artificial sequence DNA fragment amplified from salmonidae genome
gccbwwhdwrvvrcaystkbbbmwtrvwkhmratcwyrwtgcscgttrmtcrcvrmryckdbcrbyythttrwvyrchymbggkwsyyytyhwtkywtyyttwyckyhyrgskkgymydymcmrrtrsmabydmrrrrskcyvrmrmbskmrvmcyvkaystygmattccagagarymymtgyvtywkvkysmwryshyathctmthadkdatyrcwymhtkrgayrkyhraryatahrgkbrat

SEQ ID NO: 24 - DNA, 20 bp, Artificial Sequence
  Description of the artificial sequence PCR primer
accgggtctaataacccagc

SEQ ID NO: 25 - DNA, 20 bp, Artificial Sequence
  Description of the artificial sequence PCR primer
atgataatgaatgggtgttc

SEQ ID NO: 26 - DNA, 445 bp, Artificial Sequence
  Description of the artificial sequence DNA fragment amplified from salmonidae genome
wcvggvtchaayaacccmvyagghatywmmtcmsaykyhgayaaaatyhcmttycaccchtactwywcmwwyaargacvyyytnggmttnvyhsyywtmcthmyykbhmtrayayymytaryhctrttckcmccmracctcctmggvgacccrgahaaytwyacvcywgcmaayccmytmrwhachcchcchcawatcaarcchgartgatayttyytattygcmtayrcmatyythcgmtcmrtycyaayaaactwggmgghgtmcthgccctmkymbymtcnrtcctvrtycthdyhvtmrtyccyhtmctmcayahmtcyaarcaacrmrsmmtraymttycgmccmctmwsccaawbmytwtwytgrvyyctrgymgcvracmthctnayhcthacmtgaatyggrrgvmwacchgtvrrmyaccchtwyayyaycatygg

SEQ ID NO: 27 - DNA, 360 bp, Artificial Sequence
  Description of the artificial sequence DNA fragment amplified from “transvertebrate primers” SEQ ID NO  1 and 2
tycchrcbccvtchaayatytchkyhtgatgraayttygghtchythytdgsmvyntgcytnatnmyhcarmtyhymachgghytaythytagchatrcactaywcmbcngacrymdmvmymgchttytcmtchrthryccayayywsycgdraygtdmahtayggctgamthatycgvwaymtncaygchaaygghgchtchwtvttyttyatytgywthtwymtdcayrthgsvcgmgghhtmtaytayggntchtwyvyhtwynhrgaracmtgaaayayhggvrthrtmcthytvytydyaryhatarymacmkchttytyrtrgghtaygthctmccvtgrggmcaaatatcatt

SEQ ID NO: 28 - DNA, 20 bp, Artificial Sequence
  Description of the artificial sequence PCR primer
aaagctctgcgcgctctacg

SEQ ID NO: 29 - DNA, 20 bp, Artificial Sequence
  Description of the artificial sequence PCR primer
ccgcggagacattcataaac

SEQ ID NO: 30 - DNA, 202 bp, Artificial Sequence
  Description of the artificial sequence DNA fragment amplified from plant genome
aaagctctgcgcgctctacgtctaraggatctgcgaatccccctgcttatactaaaactttccaaggcccgcctcatggcatccaagttgagagagataaattgaacaagtatggtcgtcccctattgggatgtactattaaacctaaattggggttatccgctaagaactatggtagagcwgtttatgaatgtctccgcgg

SEQ ID NO: 31 - DNA, 19 bp, Artificial Sequence
  Description of the artificial sequence PCR primer
aagcagatttgggtaccac

SEQ ID NO: 32 - DNA, 19 bp, Artificial Sequence
  Description of the artificial sequence PCR primer
gatttcacggaggatggtg

SEQ ID NO: 33 - DNA, 382 bp, Homo sapiens
aagcagatttgggtaccacccaagtattgactcacccatcaacaaccgctatgtatttcgtacattactgccagtcaccatgaatattgtacggtaccataaatacttgaccacctgtagtacataaaaacccaatccacatcaaaaccccctccccatgcttacaagcaagtacagcaatcaaccttcaactatcacacatcaactgcaactccaaagccacccctcacccactaggataccaacaaacctatccacccttaacagtacatagtacataaaaccatttaccgtacatagcacattacagtcaaatcccttctcgcccccatggatgacccccctcagataggggtcccttgaccaccatcctccgtgaaat

SEQ ID NO: 34 - DNA, 382 bp, Homo sapiens
aagcagatttgggtaccacccaagtattgactcacccatcaacaaccgctatgtatttcgtacattactgccagccaccatgaatattgtacagtaccataaatacttgaccacctgtagtacataaaaacccaacccacatcaaaaccccctccccatgcttacaagcaagtacagcaatcaaccttcaactatcacacatcaactgcaactccaaagcctcccctcacccactaggataccaacaaacctacccacccttaacagtacatagcacataaagccatttaccgtacatagcacattacagtcaaatcccttctcgtccccatggatgacccccctcagataggggtcccttgaccaccatcctccgtgaaat

SEQ ID NO: 35 - DNA, 382 bp, Gadus morhua
aagcagatttgggtaccacccaagtattgactcacccatcaacaaccgctatgtatttcgtacattactgccagtcaccatgaatattgtacggtaccataaatacttgaccacctgtagtacataaaaacccaatccacatcaaaaccccctccccatgcttacaagcaagtacagcaatcaaccttcaactatcacacatcaactgcaactccaaagccacccctcacccactaggataccaacaaacctatccacccttaacagtacatagtacataaaaccattcaccgtacatagcacattacagtcaaatcccttctcgcccccatggatgacccccctcagataggggtcccttgaccaccatcctccgtgaaat

SEQ ID NO: 36 - DNA, 22 bp, Gadus morhua
cgtgcttactttacatctgcaa

SEQ ID NO: 37 - DNA, 22 bp, Gadus morhua
cgtgcttactttacatctgcaa

SEQ ID NO: 38 - DNA, 22 bp, Merluccius hubbsi
cgagcctacttcacatccgcaa

SEQ ID NO: 39 - DNA, 22 bp, Gadus morhua
cgtgcttactttacatctgcaa

SEQ ID NO: 40 - DNA, 22 bp, Gadus morhua
cgtgcttactttacatctgcaa

SEQ ID NO: 41 - DNA, 22 bp, Gadus morhua
cgtgcttactttacatctgcaa

SEQ ID NO: 42 - DNA, 22 bp, Gadus morhua
cgtgcttactttacatctgcaa

SEQ ID NO: 43 - DNA, 22 bp, Merluccius hubbsi
cgagcctacttcacatccgcaa

SEQ ID NO: 44 - DNA, 22 bp, Gadus morhua
cgtgcttactttacatctgcaa

SEQ ID NO: 45 - DNA, 22 bp, Gadus morhua
cgtgcttactttacatctgcaa

SEQ ID NO: 46 - DNA, 60 bp, Bos taurus
acaacagcattctcctctgttacccatatctgccgagacgtgaactacggctgaatcatc

SEQ ID NO: 47 - DNA, 60 bp, Bos taurus; identical to SEQ ID NO: 46

SEQ ID NO: 48 - DNA, 60 bp, Bos taurus; identical to SEQ ID NO: 46

SEQ ID NO: 49 - DNA, 60 bp, Ovis aries
acaacagcattctcctctgtaacccacatttgccgagacgtgaactatggctgaattatc

SEQ ID NO: 50 - DNA, 60 bp, Bos taurus; identical to SEQ ID NO: 46

SEQ ID NO: 51 - DNA, 60 bp, Gallus gallus
tccctagccttctcctccgtagcccacacttgccggaacgtacaatacggctgactcatc

SEQ ID NO: 52 - DNA, 60 bp, Gallus gallus; identical to SEQ ID NO: 51

SEQ ID NO: 53 - DNA, 60 bp, Gallus gallus; identical to SEQ ID NO: 51

SEQ ID NO: 54 - DNA, 60 bp, Ovis aries; identical to SEQ ID NO: 49

SEQ ID NO: 55 - DNA, 60 bp, Bos taurus; identical to SEQ ID NO: 46

SEQ ID NO: 56 - DNA, 60 bp, Ovis aries; identical to SEQ ID NO: 49

SEQ ID NO: 57 - DNA, 60 bp, Sus scrofa
acaacagctttctcatcagttacacacatctgtcgagatgtaaattacggatgagttatt

SEQ ID NO: 58 - DNA, 60 bp, Sus scrofa; identical to SEQ ID NO: 57

SEQ ID NO: 59 - DNA, 60 bp, Gallus gallus; identical to SEQ ID NO: 51

SEQ ID NO: 60 - DNA, 60 bp, Ovis aries; identical to SEQ ID NO: 49

SEQ ID NO: 61 - DNA, 60 bp, Ovis aries; identical to SEQ ID NO: 49

SEQ ID NO: 62 - DNA, 60 bp, Gallus gallus; identical to SEQ ID NO: 51

SEQ ID NO: 63 - DNA, 60 bp, Sus scrofa; identical to SEQ ID NO: 57

SEQ ID NO: 64 - DNA, 60 bp, Ovis aries
acaacagcattctcctctgtaacccacatttgccgagacgtgaactatggctgaatcatc

SEQ ID NO: 65 - DNA, 60 bp, Bos taurus; identical to SEQ ID NO: 46

SEQ ID NO: 66 - DNA, 60 bp, Bos taurus; identical to SEQ ID NO: 46

SEQ ID NO: 67 - DNA, 60 bp, Gallus gallus; identical to SEQ ID NO: 49

SEQ ID NO: 68 - DNA, 60 bp, Sus scrofa; identical to SEQ ID NO: 57

SEQ ID NO: 69 - DNA, 60 bp, Gallus gallus; identical to SEQ ID NO: 51

SEQ ID NO: 70 - DNA, 60 bp, Cairina moschata
accctcgccttctcctcagtggctaacacatgtcgaaacgtccagtacggctgactcatc

SEQ ID NO: 71 - DNA, 60 bp, Bos taurus; identical to SEQ ID NO: 46

SEQ ID NO: 72 - DNA, 60 bp, Cairina moschata; identical to SEQ ID NO: 70

SEQ ID NO: 73 - DNA, 60 bp, Bos taurus; identical to SEQ ID NO: 70

SEQ ID NO: 74 - DNA, 60 bp, Sus scrofa; identical to SEQ ID NO: 57

SEQ ID NO: 75 - DNA, 60 bp, Sus scrofa; identical to SEQ ID NO: 57

SEQ ID NO: 76 - DNA, 60 bp, Bos taurus; identical to SEQ ID NO: 46

SEQ ID NO: 77 - DNA, 60 bp, Cairina moschata; identical to SEQ ID NO: 70

SEQ ID NO: 78 - DNA, 60 bp, Sus scrofa; identical to SEQ ID NO: 57

SEQ ID NO: 79 - DNA, 60 bp, Theragra chalcogramma
gaaacagccttctcatccgtagttcacatctgtcgtgatgtaaattacggctgactaatt

SEQ ID NO: 80 - DNA, 60 bp, Bos taurus; identical to SEQ ID NO: 46

SEQ ID NO: 81 - DNA, 60 bp, Gallus gallus; identical to SEQ ID NO: 49

SEQ ID NO: 82 - DNA, 60 bp, Bos taurus; identical to SEQ ID NO: 46

SEQ ID NO: 83 - DNA, 60 bp, Gallus gallus; identical to SEQ ID NO: 51

SEQ ID NO: 84 - DNA, 60 bp, Gallus gallus; identical to SEQ ID NO: 51

SEQ ID NO: 85 - DNA, 60 bp, Salmo trutta
tcaacagccttttcctctgtttgccacatttgccgagatgttagctacggctgactcatc

SEQ ID NO: 86 - DNA, 60 bp, Gallus gallus; identical to SEQ ID NO: 51

SEQ ID NO: 87 - DNA, 60 bp, Bos taurus; identical to SEQ ID NO: 46

SEQ ID NO: 88 - DNA, 60 bp, Gallus gallus; identical to SEQ ID NO: 49

SEQ ID NO: 89 - DNA, 60 bp, Theragra chalcogramma; identical to SEQ ID NO: 79

SEQ ID NO: 90 - DNA, 60 bp, Salmo trutta; identical to SEQ ID NO: 85

SEQ ID NO: 91 - DNA, 60 bp, Cairina moschata; identical to SEQ ID NO: 70

SEQ ID NO: 92 - DNA, 60 bp, Theragra chalcogramma; identical to SEQ ID NO: 79

SEQ ID NO: 93 - DNA, 60 bp, Theragra chalcogramma; identical to SEQ ID NO: 79

SEQ ID NO: 94 - DNA, 60 bp, Cairina moschata; identical to SEQ ID NO: 79

SEQ ID NO: 95 - DNA, 60 bp, Salmo trutta; identical to SEQ ID NO: 70

SEQ ID NO: 96 - DNA, 382 bp, Homo sapiens
aagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaat

SEQ ID NO: 97 - DNA, 382 bp, Homo sapiens
  modified_base (75): a, t c or g
aagcagatttgggtaccacccaagtattgactcacccatcaacaaccgctatgtatttcgtacattactgccagncaccatgaatattgtacngtaccataaatacttgaccacctgtagtacataaaaacccaatccacancaaaaccccctccccatgcttacaagcaagtacagcaatcaaccttcaactatcacacatcaactgcaactccaaagccacccctcncccactaggataccaacaaacctanccacccttaacagtacatagnacataaanccattnaccgtacatagcacattacagtcaaatcccttctcgnccccatggatgacccccctcagataggggtcccttgaccaccatcctccgtgaaat

SEQ ID NO: 98 - DNA, 20 bp, Artificial Sequence
  Description of Artificial Sequence Primer
tcatctccggtttacaagac

SEQ ID NO: 99 - DNA, 21 bp, Artificial Sequence
  Description of Artificial Sequence Primer
tgatatgaaaaaccatcgttg

SEQ ID NO: 100 - DNA, 20 bp, Artificial Sequence
  Description of Artificial Sequence Primer
taactgtggtaattctagag

SEQ ID NO: 101 - DNA, 20 bp, Artificial Sequence
  Description of Artificial Sequence Primer
actctagataacctcgggcc

SEQ ID NO: 102 - DNA, 20 bp, Artificial Sequence
  Description of Artificial Sequence Primer
agctccaatagcgtatatta

SEQ ID NO: 103 - DNA, 20 bp, Artificial Sequence
  Description of Artificial Sequence Primer
ggaagagctgggccagctgc

SEQ ID NO: 104 - DNA, 20 bp, Artificial Sequence
  Description of Artificial Sequence Primer
caggtaggtatcccacttac